(19) United States
(12) Patent Application Publication
(10) Pub. No.: US 2006/0020036 A1
(43) Pub. Date: Jan. 26, 2006

(54) Methods and compositions to promote bone homestasis

(76) Inventors: Luc Van Rompaey, Keerbergen (BE); Peter Herwig Maria Tomme, Gent (BE)

Correspondence Address: SYNNESTVEDT LECHNER & WOODBRIDGE LLP, P O BOX 592, PRINCETON, NJ, 08542-0592, US

(21) Appl. No.: 11/166,009

(22) Filed: Jun. 24, 2005

Related U.S. Application Data
(60) Provisional application No. 60/582,704, filed on Jun. 24, 2004.
(60) Provisional application No. 60/630,449, filed on Nov. 23, 2004.
(60) Provisional application No. 60/673,206, filed on Apr. 20, 2005.

(51) Int. Cl. A61K 31/18; A61K 31/195

(52) U.S. Cl. 514/567; 514/602

(57) ABSTRACT

The present invention relates to a method for promoting osteogenesis by contacting osteoblast progenitor cells with an LXR agonist. Said method is useful for the treatment or prevention of an imbalance in bone homeostasis in a subject using bone homeostasis-promoting compositions comprising an effective osteogenic stimulating amount of an LXR agonist in admixture with a pharmaceutically acceptable carrier. A further aspect is a method to produce bone tissue in vitro by contacting an LXR agonist with a population of osteoblast progenitor cells on a substrate, for a time sufficient to stimulate the generation of a matrix of bone tissue.

### CROSS-REFERENCE TO RELATED APPLICATIONS

This application claims priority to U.S. Provisional Application No. 60/582,704, filed Jun. 24, 2004; U.S. Provisional Application No. 60/630,449, filed Nov. 23, 2004; and U.S. Provisional Application No. 60/673,206, filed Apr. 20, 2005, the disclosures of which are incorporated herein by reference.

### FIELD OF INVENTION

This invention relates to the field of bone metabolism, and in particular, to methods, therapies, and compositions useful, for the prevention and treatment of diseases associated with an imbalance, or disturbance, in bone homeostasis in humans and other animals.

Bone is a dynamic tissue that is continuously being destroyed (resorbed) and rebuilt, by an intricate interplay between two distinct cell lineages: bone-forming cells, known as osteoblasts and bone-resorbing cells, known as osteoclasts. The cascade of transcription factors and growth factors involved in the differentiation or progression from progenitor cell to functional osteoclast is well established. In contrast, little is known about the factors involved in the progression of osteoblasts from progenitor cells. The mesenchymal progenitor or stem cells (MPCs) represent the starting points for the differentiation of both osteoclasts and osteoblasts. During embryonic development in vivo, bone formation occurs through two distinct pathways: intramembranous and/or endochondral ossification (see FIG. 1; taken from Nakashima and de Crombrugghe, (2003)). During intramembranous ossification, flat bones such as those of the skull or clavicles, are formed directly from condensations of mesenchymal cells. During endochondral ossification, long bones, such as limb bones, are formed from a cartilage intermediate formed during mesenchymal condensation, which intermediate is invaded during further development by endothelial cells, osteoclasts and mesenchymal cells that further differentiate into osteoblasts and osteocytes. During this latter differentiation into osteoblasts, bone alkaline phosphatase activity (BAP) is up-regulated.

A number of diseases are the direct result of a disturbance in the fine-tuned balance between bone resorption and bone formation. These diseases for the most part are skeletal diseases and inflict a large number of patients. Exemplary diseases include hypocalcaemia of malignancy, Paget's disease, inflammatory bone diseases such as rheumatoid arthritis and periodontal disease, focal osteogenesis occurring during skeletal metastases, Crouzon's syndrome, rickets, opsismodysplasia, pycnodysostosis/Toulouse-Lautrec disease, osteogenesis imperfecta, and osteoporosis. The single most prevalent bone disease is osteoporosis, which affects 1 in 5 women over 50 and 1 in 20 men over 50.

### Reported Developments

A number of treatments have been developed and made available to patients suffering from osteoporosis and related skeletal diseases. These therapeutic approaches primarily are directed to increasing net bone formation and include: hormone replacement therapy (HRT); selective estrogen receptor modulators (SERMs); bisphosphonates; and calcitonin. While these treatments slow down bone resorption, they don't abolish fracturing because the lost bone is not sufficiently replenished. Fracturing will be prevented only if bone formation is sufficiently increased. Therefore, there is great interest in identifying osteogenic pathways that enhance bone anabolism as a basis for therapeutic intervention.

Parathyroid hormone (PTH) 1-34 is the only bone anabolic therapy on the osteoporosis therapeutic market. While PTH displays bone anabolic effects when administered intermittently, it needs to be injected daily, and may have tumorgenic side effects, based on the observation that tumors form in animals treated with at PTH in high doses.

Bone morphogenetic proteins (BMPs) are another class of bone anabolic therapeutics, but have only been approved for niche markets. Receptors for the bone morphogenetic proteins have been identified in many tissues other than bone, and BMPs themselves are expressed in a large variety of tissues in specific temporal and spatial patterns. This suggests that BMPs may have effects on many tissues other than bone, potentially limiting their usefulness as therapeutic agents when administered systemically.

There is a clear need to identify additional targets that stimulate osteogenic differentiation and that can be used for the development of novel bone anabolic therapies.

The present invention is based on the discovery that certain known polypeptides, including the LXR proteins, are factors in the up-regulation and/or induction of osteogenic differentiation in bone marrow cells, and that the known agonists for these polypeptides are effective in promoting bone homeostasis.

### SUMMARY OF THE INVENTION

The present invention relates to a method for promoting osteogenesis in a population of cells including osteoblast progenitor cells, or more particularly, cell differentiation to form osteoblast cells, comprising contacting osteoblast progenitor cells with an effective osteogenic-stimulating amount of an LXR agonist. The present method may be used for the treatment or prevention of an imbalance in bone homeostasis in a subject suffering from or susceptible to said imbalance comprising administering an effective osteogenic stimulating amount of an LXR agonist to said subject. This invention relates also to a composition for use in the aforesaid method, such as a bone homeostasis-promoting composition, comprising an effective osteogenic stimulating amount of an LXR agonist in admixture with a pharmaceutically acceptable carrier. A further aspect is a method to produce bone tissue in vitro, comprising contacting an effective osteogenic stimulating amount of an LXR agonist with a population of osteoblast progenitor cells on a substrate, for a time sufficient to stimulate the generation of a matrix of bone tissue.

### BRIEF DESCRIPTION OF THE DRAWINGS

FIG. 1. Intramembranous and endochondral ossification.

FIG. 2. Principle of the osteoblast differentiation assay.

FIG. 3. Performance of the knock-in control plate in the AP assay.

FIG. 4. Dot plot representation of raw data for one FLeXeSelect screening plate.

FIG. 5. Dose-dependent up-regulation of AP activity by selected compounds.

FIG. 6. Analyzing the up-regulation of BAP-mRNA versus PLAP- or IAP-mRNA.

FIG. 7. Mineralization of primary human MPCs.

FIG. 8. Mineralization of primary human MPCs.

FIG. 9. Dose-dependent up-regulation of AP activity by the LXR agonist GW3965 in the presence of Ad-NR1H3.

FIG. 10. Dose-dependent up-regulation of AP activity by the LXR agonist T0901317 in the presence of Ad-NR1H2.

FIG. 11. Dose-dependent up-regulation of AP activity by the LXR agonist GW3965 in the presence of Ad-NR1H2.

FIG. 12. Structure of the acetyl podocarpic dimer (APD) used in this application.

FIG. 13. Dose-dependent up-regulation of AP activity by the LXR agonist APD in the presence of Ad-NR1H2 or Ad-NR1H3.

FIG. 14A-D. Ct values and relative expression levels of the genes of the present invention compared to beta-actin for cell types relevant to bone formation.

FIG. 15. NR5A2 and NR1H3+T0901317 up-regulate mRNA levels of osteogenic markers.

FIG. 16. Up-regulation of NR5A2 and NR1H3 mRNA levels by osteogenic triggers.

FIG. 17. Weight increases in calvarial skull explants induced by the positive controls Ad-BMP2 and Ad-BMP7.

FIG. 18: Weight increases in calvarial skull explants induced by T0901317.

FIG. 19: DN-RUNX2 interferes with induction of AP activity by NR5A2, NR1H3+T0901317 and ESRRG.

FIG. 20: NR5A2, NR1H3+T0901317, and ESRRG induce AP activity independent of the MPC isolate.

### DETAILED DESCRIPTION

The following terms are intended to have the meanings presented therewith below and are useful in understanding the description of and intended scope of the present invention.

The term “agonist” refers, in the broadest sense, to a ligand that stimulates the receptor to which it binds.

The term “effective amount” means that amount of a drug or pharmaceutical agent that will elicit the biological or medical response of a subject that is being sought by a medical doctor or other clinician. In particular, with regard to treating an imbalance in bone homeostasis, the term “effective osteogenic stimulating amount” is intended to mean that effective amount of an LXR agonist or prodrug of LXR agonist that will bring about a biologically meaningful increase in the ratio of osteoblasts to osteoclasts in the subject's bone tissue. A biologically meaningful increase is that increase that can be detected indirectly by means of bone density, bone strength, or other diagnostic indicia known to those skilled in the art.

The term “expression” relates to both endogenous expression and over-expression, for example, by transfection or stable transduction.

The term “LXR” includes all subtypes of this receptor as known in the prior art and corresponding genes that encode such subtypes. Specifically LXR includes LXR-alpha and LXR-beta, and an agonist of LXR should be understood to include an agonist of LXR-alpha or LXR-beta. LXR-alpha is referred to under a variety of names and for purposes of this application LXR-alpha should be understood to mean any gene referred to as LXR-alpha, LXRₐ, LXRα, RLD-1, NR1H3 or a gene with homology to accession number U22662 or a protein with homology to a protein encoded by such a polynucleotide. Similarly, LXR-beta should be understood to include any gene referred to as LXR_{b}, LXR-beta, LXRbeta, NER, NER1, UR, OR-1, R1P15, NR1H2 or a gene with homology to accession number U07132 or a protein with homology to a protein encoded by such a polynucleotide. “Homology” means sequence similarity to the extent that polynucleotides of the “homologous” sequence are able to hybridize to the LXR sequence under stringent hybridization conditions as understood by a person of skill in the art.

The term “osteogenesis” means a process that consists of several successive events, including initially the up-regulation of bone alkaline phosphatase in a cell, and calcium deposition (mineralization) which occurs in later stages of process.

The term “osteogenic differentiation” refers to any process wherein unspecialized cells in a lineage of bone-related cells become more specialized by exhibiting anabolic processes resulting in the deposition of calcium and the formation of bone tissue.

The term “pharmaceutically acceptable carrier” includes, for example, pharmaceutically acceptable carriers such as the following: solid carriers such as lactose, magnesium stearate, terra alba, sucrose, talc, stearic acid, gelatin, agar, pectin, acacia or the like; and liquids such as vegetable oils, arachis oil and sterile water, or the like. However, this listing of pharmaceutically acceptable carriers is not to be construed as limiting.

“Pharmaceutically acceptable prodrugs” as used herein refers to those prodrugs of the compounds useful in the present invention, which are, within the scope of sound medical judgment, suitable for use in contact with the tissues of patients without undue toxicity, irritation, allergic response commensurate with a reasonable benefit/risk ratio, and effective for their intended use of the compounds of the invention. The term “prodrug” means a compound that is transformed in vivo to yield an effective compound useful in the present invention or a pharmaceutically acceptable salt, hydrate or solvate thereof. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as pro-drugs. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191, (1991); Advanced Drug Delivery Reviews, H. Bundgard, 8, 1-38, (1992); J. Pharm. Sci., 77,285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference. An example of the prodrugs is an ester prodrug. “Ester prodrug” means a compound that is convertible in vivo by metabolic means (e.g., by hydrolysis) to an LXR agonist. For example an ester prodrug of a compound containing a carboxy group may be convertible by hydrolysis in vivo to the corresponding carboxy group.

The term “pharmaceutically acceptable salts” refers to the non-toxic, inorganic and organic acid addition salts, and base addition salts, of compounds of the present invention. These salts can be prepared in situ during the final isolation and purification of compounds useful in the present invention.

The term “polynucleotide” refers to nucleic acids, such, as double stranded, or single stranded DNA and (messenger) RNA, and all types of oligonucleotides. It also includes nucleic acids with modified backbones such as peptide nucleic acid (PNA), polysiloxane, and 2′-O-(2-methoxy)ethylphosphorothioate. “Derivatives of a polynucleotide” means DNA-molecules, RNA-molecules, and oligonucleotides that comprise a stretch or nucleic acid residues of the polynucleotide, e.g. polynucleotides that may have nucleic acid mutations as compared to the nucleic acid sequence of a naturally occurring form of the polynucleotide. A derivative may further comprise nucleic acids with modified backbones such as PNA, polysiloxane, and 2′-O-(2-methoxy)ethyl-phosphorothioate, non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection. “Fragment of a polynucleotide” means oligonucleotides that comprise a stretch of contiguous nucleic acid residues that exhibit substantially a similar, but not necessarily identical, activity as the complete sequence.

The term “polypeptide” relates to proteins, proteinaceous molecules, fractions of proteins, peptides, oligopeptides, and enzymes (such as kinases, proteases, GCPRs). “Derivatives of a polypeptide” relate to those peptides, oligopeptides, polypeptides, proteins and enzymes that comprise a stretch of contiguous amino acid residues of the polypeptide and that retain the biological activity of the protein, e.g. polypeptides that have amino acid mutations compared to the amino acid sequence of a naturally-occurring form of the polypeptide. A derivative may further comprise additional naturally occurring, altered, glycosylated, acylated or non-naturally occurring amino acid residues compared to the amino acid sequence of a naturally occurring form of the polypeptide. It may also contain one or more non-amino acid substituents compared to the amino acid sequence of a naturally occurring form of the polypeptide, for example a reporter molecule or other ligand, covalently or non-covalently bound to the amino acid sequence. “Fragment of a polypeptide” relates to peptides, oligopeptides, polypeptides, proteins and enzymes that comprise a stretch of contiguous amino acid residues, and exhibit substantially a similar, but not necessarily identical, functional activity as the complete sequence.

The term “solvate” means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “subject” includes humans and other mammals.

The term “treating” refers to alleviating the disorder or condition to which the term “treating” applies, including one or more symptoms of such disorder or condition. The related term “treatment,” as used herein, refers to the act of treating a disorder, symptom, or condition, as the term “treating” is defined above.

### THE METHODS OF THE PRESENT INVENTION

The present invention relates to methods for increasing and/or inducing osteogenic differentiation, said method comprising contacting (1) a population of cells expressing a polypeptide encoded by the LXR target gene identified in Table 1 below as NR1H3, or a 5 functional fragment or derivative thereof; with (2) an LXR agonist; and (3) thereby increasing the level of osteogenic differentiation in said population of cells. The present inventors prepared Table 1 below from the results obtained from the screening studies described further below.

TABLE 1
List of identified target genes.

| | | | GenBank | GenPept |
|---|---|---|---|---|
| | | | accession | accession |
| Gene symbol | Gene description | Class | (DNA) | (Protein) |
| ADORA2A | adenosine A2a receptor | GPCR | NM_000675 | NP_000666 |
| NR1H3 | nuclear receptor subfamily 1, group H, | NHR | NM_005693 | NP_005684 |
| | member 3 | | | |
| HSU93553/NR5A2 | alpha 1-fetoprotein transcription factor | NHR | U93553 | AAD03155 |
| | (hFTF) | | NM_003822 | NP_003813 |
| | | | NM_205860 | NP_995582 |
| GPR52 | G protein-coupled receptor 52 | GPCR | NM_005684 | NP_005675 |
| RE2/GPR161 | G protein-coupled receptor 161 | GPCR | NM_007369 | NP_031395 |
| | | | NM_153832 | NP_722561 |
| | | | 3273814CA2 | 3273814CA2 |
| GPR65 | G protein-coupled receptor 65 | GPCR | NM_003608 | NP_003599 |
| ESRRG | estrogen-related receptor gamma | NHR | NM_001438 | NP_001429 |
| | | | NM_206594 | NP_996317 |
| | | | NM_206595 | NP_996318 |
| GPR12 | G protein-coupled receptor 12 | GPCR | NM_005288 | NP_005279 |
| MC5R | melanocortin 5 receptor | GPCR | NM_005913 | NP_005904 |
| AVPR2 | arginine vasopressin receptor 2 | GPCR | NM_000054 | NP_000045 |
| | (nephrogenic diabetes insipidus) | | | |
| DRD1 | dopamine receptor D1 | GPCR | NM_000794 | NP_000785 |
| NR1H2 | nuclear receptor subfamily 1, group H, | NHR | NM_007121 | NP_009052 |
| | member 2 | | | |

Methods Used to Identify Relationship between LXR and Osteogenic Differentiation

The above-identified osteogenic differentiation-related target genes were identified using a so-called ‘knock-in’ library in the following manner. Using recombinant adenoviruses, the present inventors tranduced cDNA molecules coding for a specific natural gene and gene product into cells. Each cDNA introduced into each separate subpopulation of cells induced the expression and activity of the corresponding gene and gene product in a cell. By identifying a cDNA that induces or increases osteogenic differentiation, a direct link is made to the corresponding target gene. This target gene is subsequently used in methods for identifying compounds that can be used to activate or stimulate osteogenic differentiation, at binding affinity of at most 10 micromolar. Indeed, compounds that are known to bind to target genes used in this screen were found to increase osteogenic differentiation of cells, demonstrating the role of these target genes in this process. This method was used to identify the polypeptides, including the LXR receptor, as involved in the process of osteoblast differentiation, and the use of agonists thereof to promote or induce osteoblast differentiation.

The population of cells, in which osteoblast differentiation is promoted, is preferably any undifferentiated cell type or cell types. Undifferentiated cells are pluripotent cells that are in an early stage of specialization, i.e., which do not yet have their final function and can be induced to form almost any given cell type. Such cells are especially blood cells and cells present in bone marrow, as well as cells derived from adipose tissue. In addition, cells that can still be differentiated into mesenchymal precursor cells are contemplated in the present invention, such as, for example, totipotent stem cells such as embryonic stem cells.

The polypeptide used in the knock-in library and that provided the basis for the present invention (using an LXR agonist) is in a class of nuclear hormone receptors (NHR). By way of background, lipophilic hormones such as steroids, retinoids, thyroids, and vitamin D₂ modulate gene transcription inside the cell. A steroid hormone, for example, will enter the cell and bind to its complementary receptor, initiating a complex cascade of events. The hormone-receptor complex forms dimers, which bind to a DNA sequence called the hormone response element (HRE). This binding activates, or in some cases inhibits, transcription of the appropriate gene. As such, the activity of NHRs can also be determined with a reporter gene under the control of a promoter that contains the appropriate Hormone Receptor Element (HRE).

Another of the polypeptides used in the knock-in library is a G-Protein Coupled Receptor (GPCR), wherein the expression and/or activity of said GPCR is measured by determining the level of any one of the second messengers cyclic AMP, Ca²⁺ or both. Preferably, the level of the second messenger is determined with a reporter gene under the control of a promoter that is responsive to the second messenger. More preferably, the promoter is a cyclic AMP-responsive promoter, an NF-KB responsive promoter, or a NF-AT responsive promoter. In another preferred embodiment, the reporter gene is selected from the group consisting of: alkaline phosphatase, GFP, eGFP, dGFP, luciferase and b-galactosidase.

One method to measure osteogenic differentiation, and found useful in the screen, determines the expression level of certain proteins that are involved in bone-morphogenesis and that are induced during the differentiation process, such as alkaline phosphatase, type-1 collagen, osteocalcin and osteopontin. The activity levels of these marker proteins can be measured through assays using specific substrates. For instance, the bone alkaline phosphatase (BAP, or bone AP) activity can be measured by adding a methylumbelliferyl heptaphosphate (MUP) solution to the cells. The fluorescence generated upon cleavage of the MUP substrate by the AP activity is measured on a fluorescence plate reader, as outlined in the examples given below. The expression of the target genes can also be determined by methods known in the art such as Western blotting using specific antibodies, or ELISAs using specific antibodies directed against the target genes. Alternatively, one can analyse the mRNA expression levels in cells, using methods known in the art like Northern blotting and quantitative real-time PCR.

Upon incubation with an agonist compound, osteogenic differentiation promotion may be monitored by the agonist's induction of the expression or activity of a marker protein. Although induction of protein expression levels may vary from an increase of a few percent to two, three or four orders of magnitude higher, induction of protein expression of at least twofold (or more) in a patient (in vivo) is a preferred level. A preferred induction of said expression and/or activity is therefore comparable to an induction of 100% (or more) in vivo. It can however not be excluded that levels found in vitro do not perfectly correlate with levels found in vivo, such that a slightly reduced level in vitro may still result in a higher induction in vivo when the agonist compound is applied in a therapeutic setting. It is therefore preferred to have induced in vitro levels of at least 20%, more preferably more than 50%, even more preferably more than 100%, which would mean a twofold induction of the expression or activity of the osteogenic marker protein.

For screening of a compound that influences the osteogenic differentiation of cells by binding to any of the target polypeptides listed in Table 1, or a derivative, or a fragment thereof, libraries of compounds can be used such as peptide libraries (e.g. LOPAP™, Sigma Aldrich), lipid libraries (BioMol), synthetic compound libraries (e.g. LOPAC™, Sigma Aldrich) or natural compound libraries (Specs, TimTec).

The binding affinity of the compound with the polypeptide or polynucleotide can be measured by methods known in the art, such as using surface plasmon resonance biosensors (Biacore), by saturation binding analysis with a labeled compound (e.g. Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer.

The binding affinity of compounds can also be expressed in dissociation constant (Kd) or as IC₅₀ or EC₅₀. The IC₅₀ represents the concentration of a compound that is required for 50% inhibition of binding of another ligand to the polypeptide. The EC₅₀ represents the concentration required for obtaining 50% of the maximum effect in any assay that measures receptor function. The dissociation constant, Kd, is a measure of how well a ligand binds to the polypeptide, it is equivalent to the ligand concentration required to saturate exactly half of the binding-sites on the polypeptide. Compounds with a high binding affinity have low Kd, IC₅₀ and EC₅₀ values, i.e. in the range of 100 nM to 1 pM; a moderate to low affinity binding relates to a high Kd, IC₅₀ and EC₅₀ values, i.e. ᵢₙ the micromolar range. Binding affinities may be determined in in vivo settings as well as in in vitro settings.

The induction of osteogenic differentiation of cells may be achieved in different ways. The compounds useful in the present invention may target the polypeptides directly and induce or stimulate their activity. These compounds may also target the transcription/translation machinery involved in the transcription and/or translation of the polypeptide from its encoding nucleic acid.

The compounds may furthermore target their respective DNAs and mRNAs thereby inducing the occurrence of the polypeptide and thereby their activity. It is thus to be understood that the compounds that are identified by using the methods of the present invention may target the expression, and/or the activity of the polypeptides at different levels, finally resulting in the alteration of the osteogenic differentiation of cells. The agonist compounds of the present invention may function in accordance with any one of these mechanisms.

A preferred aspect of the present invention comprises the contacting of said population of cells with an LXR agonist, or a mixture thereof. The term “LXR agonist” means a compound that up-regulates (i.e. activates or stimulates) LXR receptor activity and/or concentrations thereof in a cell, and should be understood to include an agonist or partial agonist of LXR. The agonist may be selective for LXR-alpha or LXR-beta, or it may have mixed binding affinity for both LXR-alpha and LXR-beta. Particularly, compounds within the scope of this invention include those that have greater selectivity as determined by binding affinity for LXR-alpha and/or LXR-beta receptors than they have for each of the PPAR-alpha, gamma and delta receptors. More particularly, the compounds included within the scope of this invention have an IC₅₀ less than or equal to 100 nM for at least one of either the LXR-alpha or LXR-beta receptors, and have an IC₅₀ equal to or greater than 1 micromolar for each of the PPAR-alpha, PPAR-gamma, and PPAR-delta receptors, and even more particularly they have an IC₅₀ equal to or greater than 10 micromolar for each of the PPAR-alpha, PPAR-gamma and PPAR-delta receptors. For example, the selectivity of suitable LXR receptor agonists can be determined from IC₅₀ results obtained employing the LXR radioagonist competition scintillation proximity assays described in published U.S. patent application 20030086923, and from PPAR competition binding assays described in Berger J, et al., Novel peroxisome proliferator-activated receptory (PPAR-gamma) and PPAR-delta agonists produce distinct biological effects, J Biol Chem 274: 6718-6725 (1999), herein incorporated by reference in its entirety.

Exemplary LXR agonists are disclosed in PCT publications WO224632 and WO03082198, which disclose derivatives of diarylalkylaminoalkoxy2-phenyl acetic acid, more specifically, 2-(3-(3-(N-(2-chloro-3-(trifluoromethyl)benzyl)-N-(2,2-diphenylethyl)amino) propoxy)phenyl) acetic acid; PCT publication WO0182917, and UA 20040018560, which disclose the benzenesulfonamides, N-(2,2,2-Trifluoroethyl)-N-[4-[2,2,2-trifluoro-1-hydroxy-1-(trifluoromethyl)ethyl]phenyl]-benzenesulfonamide, and N-(methyl)-N-[4-[2,2,2-trifluoro-1-hydroxy-1-(trifluoromethyl)ethyl]phenyl]-benzenesulfonamide; U.S. Pat. No. 6,645,955, which discloses steroidyl LXR agonists, including for example, 3-beta-hydroxy-5-cholesten-25(R)-26-carboxylic acid; UA 20030086923, which discloses LXR agonists, including for example, (4,5-dihydro-1-(3-(3-trifluoromethyl-7-propyl-benzisoxazol-6-yloxy)propyl)-2,6-pyrimidinedione); UA 20030125357, which discloses derivatives of 10β-podocarpane, more specifically (4β, 5α)-12-hydroxy-N-[(1-phenylcyclobutyl)methyl]podocarpa-8,11,13-trien-16-amide; UA 20040072868, which discloses substituted aminopropoxyaryl derivatives, more specifically 2-(3-{3-[[2-chloro-3-(trifluoromethyl)benzyl](2,2-diphenylethyl)amino]propoxy}phenyl)acetamide; UA 20030073614, N-(2,2,2-trifluoroethyl)-N-[4(2,2,2-trifluoro-1-hydro-xy-1-trifluoromethylethyl)-phenyl]-benzene sulfonamide; PCT publication WO2004001002, [6a-hydroxy bile acid or an oxycholestorol compound]; PCT publication WO03090732, which discloses a genus of compounds including morpholine-4-carbothioic acid (4-cyano-butyl)-[4-(2, 2,2-trifluoro-1-hydroxy-1-trifluoromethyl-ethyl)-phenyl]-amide, and 5-{(Morpholine-4-carbothioyl)-[4-(2,2,2-trifluoro-1-hydroxy-1-trifluoromethyl-ethyl)-phenyl]-amino}-pentanoic acid methyl ester; PCT publication WO03090746, which discloses 3-thiazoles, more specifically N-(2-mercapto-1,3-benzothiazol-6-yl)-N-(2-methylpropyl)-N′-[4-(trifluoromethyl) phenyl]urea; PCT publication WO03090869, which discloses a class of compounds including 3-{[5-2,2,2-Trifluoro-1-hydroxy-1-trifluoromethyl-ethyl)-4,5-dihydro-isoxazole-3-carbonyl]-amino}propionic acid ter-butyl ester,3-Methyl-2-{[5-2, 2, 2-trifluoro-1-hydroxy-1-trifluoromethyl-ethyl)-4,5-dihydro-isoxazole-3-carbonyl]-amino}-butyric acid tert-butyl ester, and N-pyridin-4-ylmethyl-N-[5-(2,2,2-trifluoro-1-hydroxy-1-trifluoromethyl-ethyl)-thiazol-2-yl] isonicotinamide; PCT publication WO03031408, which discloses tricyclic compounds, more specifically trans-8-hydroxy-9-hydro-1,2-[a,b][(1-carboxyethyl-2-N-pyrolidinyl) benzo-4,5-yl]-cis-10-methyldecalin also named 5-Hydroxy-8a-methyl-2-pyrrolidin-1-yl-4b, 5,6, 7,8, 8a, 9,10-octahydro-phenanthrene-3-carboxylic acid ethyl ester; 8-Keto-1,2-[a,b] [(1-carboxyethyl-1-N-pyrolidiny10 benzo-4,5-yl]-10-methyldecalin also named 8a-methyl-5-oxo-2-pyrrolidin-1-yl-4b, 5,6, 7,8, 8a, 9,10-octahydro-phenanthrene-3-carboxylic acid ethyl ester; and 8-hydroxy-1,2-[a, b] [(1-hydroxymethyl-1-N-pyrolidinyl) benzo-4,5-yl]-10-methyldecalin also named 6,10a-dimethyl-7-pyrrolidin-1-yl-1, 2,3, 4,4a, 9,10, 10a-octahydro-phenanthren-4-ol, also named6-Hydroxymethyl-10a-methyl-7-pyrrolidin-1-yl-1, 2,3, 4,4a, 9,10, 10a-octahydro-phenanthren-4-ol; PCT publication WO2004009091, which discloses purine derivatives, more specifically 7-(2-chloro-6-fluorobenzyl)-1,3-diethyl-8-piperidin-1-yl-3,7-dihydro-1H-purine-2,6-dione; PCT publication WO2004024161, which discloses 2-amino-4-oxoquinazolones, more specifically identified therein as TR1040001892, TR1040011382, TR1040002211 and TR1040002212; PCT publication WO2004024162, which discloses 2-amino-4-quinazolones, more specifically [MOLNAMES 3252, 6584, 7459, and 7364]; PCT publication WO2004011448, which discloses a class of compounds including more specifically 1-(3-1 [7-propyl-3-(neopentyl)-1,2-benzisoxazol-6-yl]oxy}propyl)pyrrolidine-2,5-dione; PCT publication WO03053352, which discloses a class of compounds, more specifically the group consisting of [N-methyl-N-(3-{[7-propyl-3-(trifluoromethyl)-1,2-benzisoxazol-6-yl] oxy} propyl)] isophthalic acid monoamide; N-methyl-N-(3-{[7-propyl-3-(trifluoromethyl)-1,2-benzisoxazol-6-yl] oxy} propyl) succinic acid monoamide; 4-carboxy-3,3-dimethyl-[N-methyl-N-(3-f [7-propyl-3-(trifluoromethyl)-1,2-benzisoxazol-6-yl] oxy} propyl)] butyramide; N-methyl-N-(3-{[7-propyl-3-(trifluoromethyl)-1,2-benzisoxazol-6-yl] oxy} propyl) acetamide; [N-methyl-N-(3-{[7-propyl-3-(trifluoromethyl)-1,2-benzisoxazol-6-yl]oxy} propyl)] thiophene-1,5-dicarboxylic acid monoamide; [N-methyl-N-(3-{[7-propyl-3-(trifluoromethyl)-1,2-benzisoxazol-6-yl] oxy} propyl)] pyridine 3,5-dicarboxylic acid monoamide; (N-methyl-N-(3-1 [7-propyl-3-(trifluoromethyl)-1,2-benzisoxazol-6-yl] oxy} propyl)] 2,2-dichlorocyclopropane-1,3-dicarboxylic acid monoamide; and the pharmaceutically acceptable salts and esters thereof]; PCT publication WO03045382, which discloses a class of compounds including N. N-dimethyl-4-{7-propyl-3-(trifluoromethyl)-1,2-benzisoxazol-6-ylloxy}butyramide; N-methyl-4-{7-propyl-3-(trifluoromethyl)-1,2-benzisoxazol-6-ylloxy}butyramide; N,N-Dimethyl-4-{7-propyl-3-neopentyl-1,2-benzisoxazol-6-ylloxy}butyramide; N-Methyl-4-{7-propyl-3-neopentyl-1,2-benzisoxazol-6-ylloxy}butyramide; N-Ethyl, 4-{7-propyl-3-neopentyl-1,2-benzisoxazol-6-yloxy}butyramide; N,N-Diethyl. 4-{7propyl-3-neopentyl-1,2-benzisoxazol-6-yloxy}butyramide; 4-{7-propyl-3-neopentyl-1,2-benzisoxazol-6-butyl}piperidine; N-Propyl, 4-{7-propyl-3-neopentyl-1,2-benzisoxazol-6-yloxy}butyramide; N-(2-Furyl) methyl. 4-{7-propyl-3-neopentyl-1,2-benzisoxazol-6-yloxy}butyramide; N-Butyl-4-{7-propyl-3-neopentyl-1,2-benzisoxazol-6-ylloxy}butyramide; 4-{[7-propyl-3-(trifluromethyl)-1,2-benzisoxazol-6-yl]oxy}butyramide; N-Propyl 4-{[7-propyl-3-(trifluromethyl)-1,2-benzisoxazol-6-yl]oxy}butyramide; 4-{7-propyl-3-(trifluromethyl)-1,2-benzisoxazol-6-yloxy}butyrylpiperidine; N-(4-carbomethoxyphenyl)methyl, 4-{7-propyl-3-(trifluromethyl)-1,2-benzisoxazol-6-ylloxy}butyramide; N-(4-carboxyphenyl)methyl, 4-{-propyl-3-(trifluromethyl)-1,2-benzisoxazol-6-yloxy}butyramide; N-Methyl-N-(4-carboxyphenyl) methyl 4-{7-propyl-3-(trifluromethyl)-1,2-benzisoxazol-6-yloxy}butyramide; N-(3-carbo-t-butyloxyphenyl) methyl 4-{7-propyl-3-(trifluromethyl)-1,2-benzisoxazol-6-yloxy}butyramide; N-Methyl, -N-(3-carboxyphenyl)methyl 4-{[7-propyl-3-(trifluromethyl)-1,2-benzisoxazol-6-yl]oxy}butyramide; N-(2-(carbo-t-butyloxy) methylphenyl) methyl 4-{7-propyl-3-(trifluromethyl)-1,2-benzisoxazol-6-ylloxy}butyramide; N-(3-carboxyphenyl)methyl, 4-{[7-propyl-3-(trifluromethyl)-1,2-benzisoxazol-6-ylloxy}butyramide; N-2-(carboxymethyl)phenyllmethyl, 4-{7-propyl-3-(trifluromethyl)-1,2-benzisoxazol-6-alloxy}butyramide; N-Methyl-N-2-(carboxymethyl)phenylmethyl 4-{7-propyl-3-(trifluromethyl)-1,2-benzisoxazol-6-ylloxy}butyramide; t-Butyl ester of 4-{7-propyl-3-(trifluromethyl)-1,2-benzisoxazol-6-ylloxy}butyric acid valine amide; rac 4-{7-propyl-3-(trifluromethyl)-1,2-benzisoxazol-6-yloxy}butyric acid valine amide; rac4-{7-propyl-3-(trifluromethyl)-1,2-benzisoxazol-6-ylloxy}butyric acid N-methylvaline amide; N-Methyl-N-(4-pyridyl) 4-{7-propyl-3-(trifluromethyl)-1,2-benzisoxazol-6-yloxy}butyramide; N-Methyl-N-(2-pyridyl) 4-{7-propyl-3-(trifluromethyl)-1,2-benzisoxazol-6-ylloxy}butyramide; N-(4-{7-propyl-3-(trifluoromethyl)-1,2-benzisoxazol-6-ylloxy}butanoyl)-L-alanine-t-butyl ester; and, N-methyl-N-(4-{7-propyl-3-(trifluoromethyl)-1,2-benzisoxazol-6-ylloxy}butanoyl)-L-alanine; PCT publication WO03082205, which discloses a class of compounds including 2-(3-{3-[[2-chloro-3-(trifluoromethyl)benzyl](2,2-dephenylethyl)amino]propoxy}phenyl)ethanol; [3-[4-(t-butyldimethylsilylhydroxy) but-1-ynyl] phenyllacetic acid methyl ester, {3-[4-hydroxybutyl]phenyl} acetic acid methyl ester, {3-[4-(toluene-4-sulfonyloxy)butyl]phenyl} acetic acid methyl ester, (S)-(2-chloro-3-trifluoromethyl-benzyl)-(2-phenyl-propyl)-amine, (R)-(2-chloro-3-trifluoromethyl-benzyl)-(2-phenyl-propyl)-amine, (2-chloro-3-trifluoromethyl-benzyl)-(naphthalene-1-ylmethyl)-amine, (2-chloro-3-trifluoromethyl-benzyl)(phenethyl)-amine, (2-chloro-3-trifluoromethyl-benzyl)-(benzyl)-amine, (2-chloro-3-trifluoromethyl-benzylamino)-phenyl-ethanol, 3-(3-benzyloxy-benzyl)-1, 2, 4-triazole, 3-(3-benzyloxy-benzyl)-ethoxymethyl-1,2,4-triazole, [3-(ethoxymethyl)-1, 2, 4-triazol-3-ylmethyl]-phenol, {3-[3-(3-bromo-propoxy)-benzyl]}-(ethoxymethyl)-1,2,4-triazole, (2-chloro-3-trifluoromethyl-benzyl)-(2,2-diphenyl-ethyl)-{3-[3-(ethoxymethyl)-1,2,4-triazol-3-ylmethyl-phenoxy]-propyl}-amine, 5-(3-benzyloxy-benzyl)-1, 2,3,4-tetrazole, 5-(3-benzyloxy-benzyl)-ethoxymethyl-1, 2,3,4-tetrazole, 5-(3-hydroxy-benzyl)-ethoxymethyl-1, 2,3,4-tetrazole, 5-[3-(3-bromo-propoxy)-benzyl]-(ethoxymethyl)-1,2,3,4-tetrazole, and (2-chloro-3-trifluoromethyl-benzyl)-(2,2-diphenyl-ethyl)-{3-[3-(ethoxymethyl-1, 2,3,4-tetrazol-5-ylmethyl)-phenoxy]-propyl}-amine, and pharmaceutically acceptable salts or solvates thereof; PCT publication WO03082192, which discloses substituted aminoalkyl heterocycles, more specifically 2-[2-{[2-chloro-3-(trifluoromethyl)-benzyl](2,2-diphenylethyl)amino}ethyl]-5-benzofuran acetic acid; PCT publication WO03082802, which discloses a class of compounds including (R)-2-(3-{3-[[2-chloro-3-(trifluoromethyl)benzyl](2,2-diphenylethyl)amino]-2-methyl-propoxy}-phenyl) acetic acid methyl ester; PCT publication WO2004043939, which discloses a class of compounds including 2-(3-{3-[(2-chloro-3-trifluoromethyl-benzyl)-diphenylethyl-amino]-propoxy}-phenyl)-N-(2-morpholin-4-yl-ethyl)-acetamide; PCT publication WO2004058175, which discloses a class of compounds including 3-chloro-4-(3-(7-propyl-3-trifluoromethyl-6-(4,5)-isoxazolyl)propylthio)-phenyl acetic acid; PCT publications WO0054759 and WO03074101, PCT publication WO0160818; and European Patent Application Pub. No. EP1398032, which discloses 4-oxo-quinazolines, more specifically the compound is identified as MOLNAME LN 7181, each of which disclosure of LXR agonist compounds and their methods of preparation is incorporated herein by reference.

In Vitro Methods of the Present Invention

A special embodiment of the present invention relates to a method for the in vitro production of bone tissue, comprising applying osteoblast progenitor cells on a substrate, and contacting said cells with an effective osteogenic stimulating amount of an LXR agonist for a time sufficient to stimulate the generation of a matrix of bone tissue. More specifically, this method is useful for the in vitro production of bone tissue, by applying mammalian osteoblast progenitor cells on a substrate; adding an LXR agonist; allowing the cells to undergo osteogenic differentiation and to generate bone tissue.

This in vitro produced bone tissue can be used for the provision of load-bearing implants, including joint prostheses, such as artificial hip joints, knee joints and finger joints, and maxillofacial implants, such as dental implants. It can also be used for special surgery devices, such as spacers, or bone fillers, and for use in augmentation, obliteration, or reconstitution of bone defects and damaged or lost bone. The methods of the invention are also very suitable in relation to revision surgery, i.e., when previous surgical devices have to be replaced. A further aspect of this method comprises combining a load-bearing implant (preferably coated with a matrix of bone tissue as described above) with a bone filling composition comprising a matrix as described above.

Preferred cells to use for the in vitro production of bone tissue are undifferentiated cells. Suitable undifferentiated cells are bone marrow cells, including haematopoietic cells and in particular stromal cells. The marrow cells, and especially the stromal cells are found to be very effective in the bone producing process when taken from their original environment. Undifferentiated cells are often available in large quantities, are more conveniently to use than mature bone cells, and exhibit a lower morbidity during recovery. Moreover, the undifferentiated cells can be obtained from the patient for whom the implant is intended. The bone resulting from these cells is autologous to the patient and thus no immune response will be induced.

The undifferentiated cells can be directly applied to the substrate or they can advantageously be multiplied in the absence of the substrate before being applied on the substrate. In the latter mode, the cells are still largely undifferentiated. Subsequently, the cells are allowed to differentiate by adding the LXR agonist as described herein, or another type of agonist that has been identified using any of the methods described herein.

Bone formation can be optimized by variation in mineralization, both by inductive and by conductive processes. In this way, matrices up to 100 μm in thickness can be produced. The cells are cultured for a time sufficient to produce a matrix layer, for example, a matrix layer having a thickness of at least 0.5 micrometer (μm), preferably between 1 and 100 μm, and more preferably between 10 and 50 μm. The cells may be contacted with the culture medium for any length of time.

The production of the matrix, when applied on a substrate, results in a continuous or quasi-continuous coating covering the substrate for at least 50% of its surface area. The substrate on which the undifferentiated cells can be applied and cultured can be a metal, such as titanium, cobalt/chromium alloy or stainless steel, a bioactive surface such as a calcium phosphate, polymer surfaces such as polyethylene, and the like.

In another embodiment, the present invention relates to cells that have undergone osteoblast differentiation by treatment with compounds as disclosed herein and identifiable according to any one of the methods described herein.

Methods of Therapy and Pharmaceutical Compositions

The present inventors discovered that the polypeptides listed in Table 1 are involved in the osteogenic differentiation process. Accordingly, the present invention relates to the link between certain polypeptides present in the cell with osteogenic differentiation of cells, some of which are closely related to the onset, occurrence, and substantiation of metabolic bone diseases. Accordingly, the present invention relates not only to the compounds that may be used for targeting these polypeptides (many of which are known in the art) but also to the use of such compounds for therapeutic purposes related to diseases of bone metabolism. For the compounds that are already known to bind to these polypeptides, the use thereof in the present invention is a new (medical) use.

A preferred aspect of the present invention relates to a method for the treatment or prevention of an imbalance in bone homeostasis comprising administering an effective osteogenic stimulating amount of an LXR agonist to a subject suffering from or susceptible to said imbalance. Such imbalance is characterized by a reduction in the ratio of osteoblasts to osteoclasts in the bone tissue of a subject. More particularly, this reduction is in the ratio of osteoblasts that are effective in mineralizing the bone matrix relative to the osteoclasts effectively resorbing bone minerals, specifically calcium.

The present method is useful for the treatment of subjects susceptible to or suffering from hypocalcaemia (of malignancy), Paget's disease, rheumatoid arthritis, periodontal disease, focal osteogenesis occurring during skeletal metastases, Crouzon's syndrome, rickets, opsismodysplasia, pycnodysostosis/Toulouse-Lautrec disease, osteogenesis imperfecta and/or osteoporosis. The most preferred method of this invention comprises the administration of the LXR agonist in pharmaceutically effective amounts to a subject susceptible and/or suffering from osteoporosis.

The LXR agonists useful in the present invention are effective in promoting the differentiation of osteoblast progenitor cells, including mesenchymal stem cells, into osteoblasts in said subject's bone marrow thereby increasing the ratio of osteoblasts to osteoclasts. A preferred class of LXR agonist comprises a derivative of a diarylalkylaminoalkoxy2-phenyl acetic acid or a pharmaceutically acceptable salt, solvate or hydrate thereof. An exemplary preferred compound is the LXR agonist, 2-(3-(3-(N-(2-chloro-3-(trifluoromethyl)benzyl)-N-(2,2-diphenylethyl)amino)propoxy)phenyl)acetic acid (GW3965), a prodrug thereof, or a pharmaceutically acceptable salt, solvate or hydrate thereof. Another preferred LXR agonist is N-(methyl)-N-[4-[2,2,2-trifluoro-1-hydroxy-1-(trifluoromethyl)ethyl]phenyl]-benzenesulfonamide, a prodrug thereof, or a pharmaceutically acceptable salt, solvate or hydrate thereof. A further preferred LXR agonist is N-(2,2,2-trifluoroethyl)-N-[4-[2,2,2-trifluoro-1-hydroxy-1-(trifluoromethyl)ethyl] phenyl]-benzenesulfonamide (T0901317), a prodrug thereof, or a pharmaceutically acceptable salt, solvate or hydrate thereof.

Administering of the LXR agonist to the subject patient includes both self-administration and administration by another person. The patient may be in need of treatment for an existing disease or medical condition, or may desire prophylactic treatment to prevent or reduce the risk for diseases and medical conditions affected by a disturbance in bone metabolism. The LXR agonist may be delivered to the subject patient orally, transdermally, via inhalation, injection, nasally, rectally, or via a sustained release formulation.

A preferred therapeutically effective amount of the LXR agonist to administer to a subject patient is about 0.01 mg/kg to about 10 mg/kg administered from once to three times a day. For example, an effective regimen of the present method may administer about 5 mg to about 1000 mg of said LXR agonist from once to three times a day. It will be understood, however, that the specific dose level for any particular subject patient will depend upon a variety of factors including the age, body weight, general health, sex, diet, time of administration, route of administration, rate of excretion, drug combination and the severity of the particular osteoblast deficiency. A consideration of these factors is well within the purview of the ordinarily skilled clinician for the purpose of determining the therapeutically effective or prophylactically effective dosage amount needed to prevent, counter, or arrest the progress of the condition.

A preferred regimen of the present method comprises the administration of an effective osteoblast differentiation-stimulating amount of a LXR agonist to a subject patient for a period of time sufficient to reestablish normal bone homeostasis and thereafter to maintain such homeostasis. A special embodiment of the method comprises administering of an effective osteoblast differentiation-stimulating amount of a LXR agonist to a subject patient susceptible to the development of osteoporosis to prevent the onset of osteoporosis.

Another aspect of the present invention relates to a bone homeostasis-promoting composition comprising an effective osteogenic-stimulating amount of an LXR agonist in admixture with a pharmaceutically acceptable carrier.

The invention relates to the use of an LXR agonist in the manufacture of a medicament for the treatment of bone-related diseases. One preferred medicament is useful for the treatment of osteoporosis.

Some of the LXR agonists useful in the present invention are basic, and such agonists are useful in the form of the free base or in the form of a pharmaceutically acceptable acid addition salt thereof. Acid addition salts are a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. In particular, acid addition salts can be prepared by separately reacting the purified compound in its free base form with a suitable organic or inorganic acid and isolating the salt thus formed. Pharmaceutically acceptable salts within the scope of the invention include those derived from mineral acids and organic acids. Exemplary acid addition salts include the hydrobromide, hydrochloride, sulfate, bisulfate, phosphate, nitrate, acetate, oxalate, valerate, oleate, palmitate, quinates, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts. See, for example S. M. Berge, et al., “Pharmaceutical Salts,” J. Pharm. Sci. 66, 1-19 (1977), which is incorporated herein by reference.

Where the LXR agonist compounds useful in the present invention are substituted with an acidic moiety, base addition salts may be formed and are simply a more convenient form for use, and in practice, use of the salt form inherently amounts to use of the free acid form. The bases which can be used to prepare the base addition salts include preferably those which produce, when combined with the free acid, pharmaceutically acceptable salts, that is, salts whose cations are non-toxic to the patient in pharmaceutical doses of the salts, so that the beneficial inhibitory effects inherent in the free base are not vitiated by side effects ascribable to the cations. Base addition salts can also be prepared by separately reacting the purified compound in its acid form with a suitable organic or inorganic base derived from alkali and alkaline earth metal salts and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine.

The LXR agonists or prodrugs of LXR agonists used according to the present invention, whether administered separately or as a pharmaceutical composition of the present invention, can be formulated according to known methods for preparing pharmaceutically useful compositions.

Pharmaceutical compositions based upon LXR agonists may be formulated for a variety of routes of administration, including, for example, orally-administrable forms such as tablets, capsules or the like, or via parenteral, intravenous, intramuscular, transdermal, buccal, subcutaneous, suppository, or other route. In certain pharmaceutical dosage forms, certain of the present LXR agonists may be more appropriate than other compounds, depending upon the route of administration and the targeted site within the patient. The compositions of the invention can be formulated so as to provide quick, sustained or delayed release of the active ingredient after administration to the patient by employing procedures known in the art. Formulations are described in a number of sources that are well known and readily available to those skilled in the art. For example, Remington's Pharmaceutical Science (Martin E W [1995] Easton Pa., Mack Publishing Company, 19.sup.th ed.) describes formulations, which can be used in connection with the present invention.

In preparing pharmaceutical compositions in oral dosage form according to the present invention, any one or more of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, tablets or capsules may be enteric-coated or sustained release by standard techniques.

Where appropriate, dosage unit formulations for oral administration can be microencapsulated. The formulation can also be prepared to prolong or sustain the release as for example by coating or embedding particulate material in polymers, wax, or the like.

Formulations suitable for parenteral administration include, for example, aqueous sterile injection solutions, which may contain antioxidants, buffers, bacteriostats, and solutes that render the formulation isotonic with the blood of the intended recipient; and aqueous and nonaqueous sterile suspensions, which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the present invention can include other agents conventional in the art having regard to the type of formulation in question.

Topical pharmaceutical compositions may be in the form of a solution, cream, ointment, mousse, gel, lotion, powder or aerosol formulation adapted for application to the skin. Topical preparation containing the LXR agonists or prodrugs of LXR agonists can be admixed with a variety of carrier materials or pharmaceutically acceptable excipients well known in the art. When the excipient serves as a diluent, it can be a solid, semi-solid, or liquid, which acts as a vehicle, carrier, or medium for the active ingredient. Thus, the compositions can be in the form of powders, suspensions, emulsions, solutions, syrups, alcoholic solutions, ointments, topical cleansers, cleansing creams, skin gels, skin lotions, mousses, roll-ons, aerosol or non-aerosol sprays in cream or gel formulations and soft gelatin capsules.

For parenteral formulations, the carrier may comprise sterile water or aqueous sodium chloride solution in combination with other ingredients that aid dispersion, such as ethanol and other pharmaceutically acceptable solvents. Of course, where solutions are to be used and maintained as sterile, the compositions and carrier must also be sterilized. Injectable suspensions may also be prepared, in which case appropriate liquid carriers, suspending agents and the like may be employed.

### EXAMPLE A

### Oral Tablet Formulation

Tablets are prepared comprising the following ingredients in parts by weight:

| | | |
|---|---|---|
| GW3865 (as K+ salt) | 10 | parts |
| lactose monohydrate | 64 | parts |
| corn starch | 20 | parts |
| polyvinylpyrrolidone | 5 | parts |
| (Polyvidone K 30) | | |
| magnesium stearate | 1 | part |

The active compound, lactose monohydrate and corn starch are sieved through a 0.63 mm sieve, mixed in a cube blender for 10 minutes, granulated with an aqueous solution of polyvinylpyrrolidone in water (50 g in 200 ml of water), dried, sized through an 0.8 mm sieve together with the magnesium stearate, mixed and pressed into tablets having a diameter of 6 mm and an average weight of 100 mg using a conventional tablet press such as a Korsch EK 0 eccentric press.

### EXAMPLE B

### Oral Liquid Formulation

An orally administrable liquid formulation is prepared comprising the following ingredients in parts by weight:

| | | |
|---|---|---|
| T0901317 | 10 | parts |
| potassium sorbate | 10 | parts |
| sodium citrate | 6 | parts |
| citric acid | 2 | parts |
| sodium chloride | 2 | parts |
| sucrose | 200 | parts |

Sufficient water is used to achieve a solution volume containing 10 g T0901317 per liter of solution. The solid ingredients are all dissolved in water, filtered through a 0.23 micron membrane and filled into bottles. 1 ml of the resulting solution contains 10 mg of T0901317. Individual dosing can be achieved by administering individual volumes of the solution to the patient.

### EXAMPLE 3

### Nasal Spray Formulation

A nasal spray formulation is prepared comprising the following ingredients in parts by weight:

| | | |
|---|---|---|
| T0901317 | 80 | parts |
| benzalkonium chloride | 1 | part |
| polyoxyethylene (20) sorbitan | | |
| monooleate (Polysorbate 80) | 80 | parts |
| sodium carboxymethylcellulose | 80 | parts |
| (Tylose.TM. C 30) | | |
| disodium hydrogen phosphate | 72 | parts |
| sodium dihydrogen phosphate | 32 | parts |
| dextrose | 240 | parts |

Sufficient purified water is used to achieve a volume containing 10 g T0901317 per liter of solution. The solid ingredients were all dissolved in the water, filtered through a 0.5 micron membrane and, filled into bottles topped by a spray pump with a volumetric dispensing chamber of 100 microliters for nasal administration.

Suppositories containing LXR agonist or a prodrug of LXR agonist may be prepared by melting 95 g of a commercially available suppository base at about 40 to 45 degree C., adding 3 g of salicylic or mandelic acid, followed by adding, while stirring, 2 g of the LXR agonist ingredient and pouring the mixture into molds.

Detailed Experimental Study Linking LXR Agonists and Osteoblast Differentiation

### EXAMPLE 1

### Screening of FLeXSelect Libraries for Modulators of Endogenous Alkaline Phosphatase in Primary Human MPCs

Materials:

Adenoviral constructs:

Ad-BMP2: Described in WO 03/018799
Ad-eGFP: Referred to as pIPspAdApt6-EGFP in WO 02070744
Ad-LacZ: Referred to as pIPspAdApt6-lacZ in WO 02070744
Ad-empty: Referred to as empty virus (generated from pIPspAdApt 6) in WO 02070744
Ad-hCAR: hCAR cDNA is isolated using a PCR methodology. The following hCAR-specific primers are used: HuCAR_for 5′-GCGAAGCTTCCATGGCGCTCCTGCTGTGCTTCG-3′ and HuCAR_rev 5′-GCGGGATCCATCTATACTATAGACCCATCCTTGCTC-3′. The hCAR cDNA is PCR amplified from a HeLa cell cDNA library (Quick clone, Clontech). A single fragment of 1119 bp is obtained and digested with the HindIII and BamHI restriction enzymes. pIPspAdapt6 vector (WO99/64582) is digested with the same enzymes, gel-purified and used to ligate to the digested PCR hCAR fragment. AdC20 (Ad5/Ad51) viruses are generated as described in WO02/24933
H4-2: described as DLL4_v1 in WO03/018799
H4-291: SPINT1_v1. cDNA is prepared from RNA isolated from human placenta and cloned in the pIPspAdapt 6 plasmid using SalI-NotI restriction sites as described in WO02/070744. The protein encoded by H4-291 is identical to NP_{—}003701.
   Principle of the Assay

Mesenchymal progenitor cells (MPCs) differentiate into osteoblasts in the presence of appropriate factors (e.g. BMP2). An assay to screen for such factors is developed by monitoring the activity of alkaline phosphatase (AP) enzyme, an early marker in the osteoblast differentiation program. MPCs are seeded in 384 well plates and simultaneously co-infected one day later with adenoviruses encoding the human coxsackie and adenovirus receptor (hCAR; Ad-hCAR) and individual adenoviruses (Ad-cDNA) from the arrayed adenoviral knock-in collection containing cDNA sequences corresponding to genes from “drugable” classes like GPCR's, kinases, proteases, phosphodiesterases and nuclear hormone receptors (the FLeXSelect collection). The majority of these cDNAs are obtained by a PCR-based approach. Briefly, PCR primers are designed for amplification of the complete open reading frame from ATG start codon to the stop codon of drugable genes, based on sequence data present in the RefSeq database. Primers are mixed in an arrayed format at a PCR ready concentration in 96 well plates. As a template for the PCR reactions, placental, fetal liver, fetal brain and spinal cord cDNA libraries are used (from Invitrogen or Edge Biosystems). For the genes encoded by a single exon, PCR reactions are also performed on human genomic DNA. After the amplification reactions, the PCR products are purified with a 96-well PCR clean-up system (Wizard magnesil, Promega, Madison, Wis., USA), digested with the appropriate restriction enzymes (AscI, NotI or SalI restriction sites are included in the primers) and directly cloned into the adenoviral adapter plasmid pIspAdAdapt-10-Zeo (described in U.S. Pat. No. 6,340,595) using DNA ligation kit version 2 (TaKaRa, Berkeley, CA, USA). After a transformation and selection step, multiple clones per gene, one of which is sequence verified, are used for the preparation of plasmid DNA and subsequent generation of adenovirus according to the procedure described in WO99/64582.

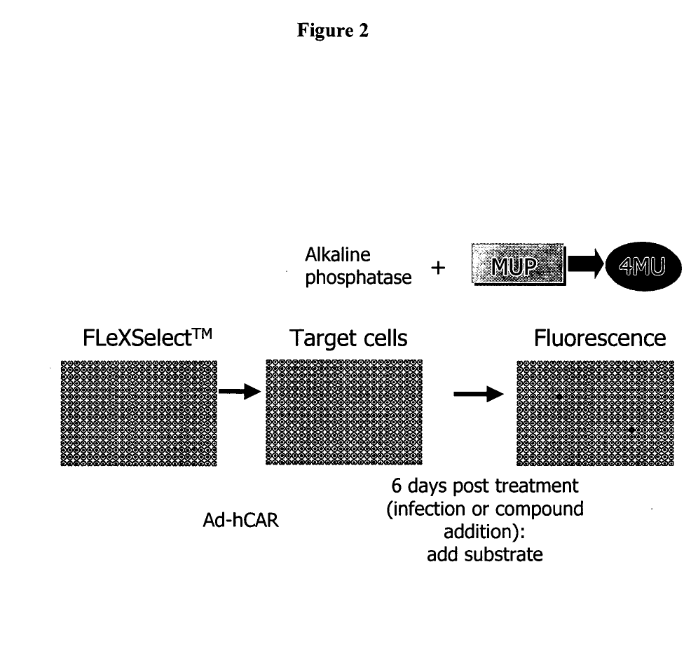

Co-infection with AdC20-hCAR (MOI 250) increases the AdC01-cDNA infection efficiency. Cellular AP activity is determined 6 days after the infection (or ligand addition—see below). The principle of the assays is depicted in FIG. 2. Mesenchymal stem cells derived from bone marrow are infected with the FLeXSelect™ cDNA library viruses in the presence of Ad5C15-hCAR or Ad5C20-hCAR virus. Six days after the start of infection or treatment with a ligand, endogenous alkaline phosphatase activity is measured following addition of 4-methylumbelliferyl heptaphosphate (MUP) substrate.

Development of the Assay

MPCs are isolated from bone marrow of healthy volunteers, obtained after informed consent (Cambrex/Biowhittaker, Verviers, Belgium).

In a series of experiments carried out in 384 well plates, several parameters are optimized: cell seeding density, multiplicities of infection (MOI) of control viruses (Ad-BMP2 or Ad-eGFP), MOI of Ad-hCAR, duration of infection, toxicity, infection efficiency (using Ad-eGFP) and the day of readout.

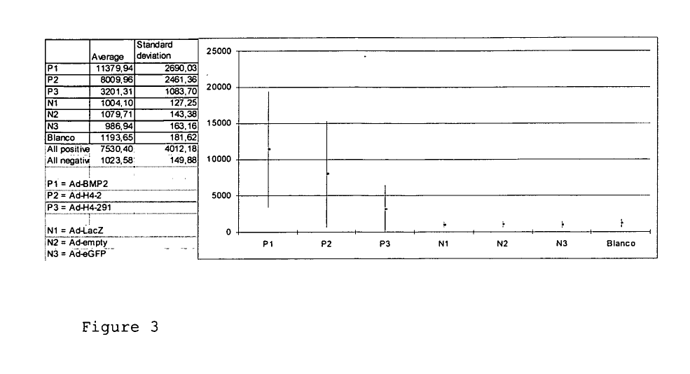

The following protocol resulted in the highest dynamic range for the assay with the lowest standard deviation on the background signal: MPCs are seeded on day 0 at 1000 cells per well of a 384 well plate and co-infected the next day using a mix of AdC20-hCAR and 2 μl of Ad-control-viruses. The stocks of the Ad-control-viruses are generated in 96 well plates (control plate). The 2 μl volume corresponds to a theoretical MOI of 5000. Controls are: P1=Ad-BMP2; P2=Ad-H4-2; P3=Ad-H4-291; N1=Ad-LacZ; N2=Ad-empty; N3=Ad-eGFP. Up-regulation of alkaline phosphatase is read at 6 days post infection (6 dpi): 15 μl 4-Methylumbelliferyl-phosphate (MUP, Sigma) is added to each well, the plates are incubated for 15 min at 37° C. and monitored for AP activity using a fluorescence plate reader (Fluostar, BMG). Pipetting of viruses from 96 well plates (containing control viruses) or 384 well plates (containing FleXSelect viruses (see next paragraph)) into 384 well plates containing MPCs is performed using robotics (96/384 channel dispenser Tecan Freedom 200 equipped with TeMO96, TeMO384 and RoMa, Tecan AG, Switzerland). FIG. 3 shows results of the automated screening procedure using the control plate. The mean and standard deviations of the negative controls (N-1-N3) are used to calculate a cut-off for hit analysis. The positive controls (P1, P2, P3) routinely scored in 80-100% of the infected wells (FIG. 3). The negative control viruses routinely scored in 0-5% of the infected wells (FIG. 3).

FleXSelect Libraries

Galapagos Genomics NV (Galapagos) built proprietary knock-in (FLeXSelect) arrayed adenoviral libraries encoding most of the drugable genes present in the human genome. The alkaline phosphatase assay is useful to screen viruses from the FLeXSelect collection (Ad-cDNA) for those classes of drugable targets that can be activated by a compound, e.g. G-protein coupled receptors (GPCRs) and nuclear hormone receptors (NHRs).

For a subset of the Ad-GPCRs present in the FLeXSelect library a matching collection of ligands is prepared in 96 and 384 well plates, such that robotics can be used to pipet a matching pair of Ad-GPCR and ligand from the respective stocks in one well of a 384 well plate containing MPCs.

Screening

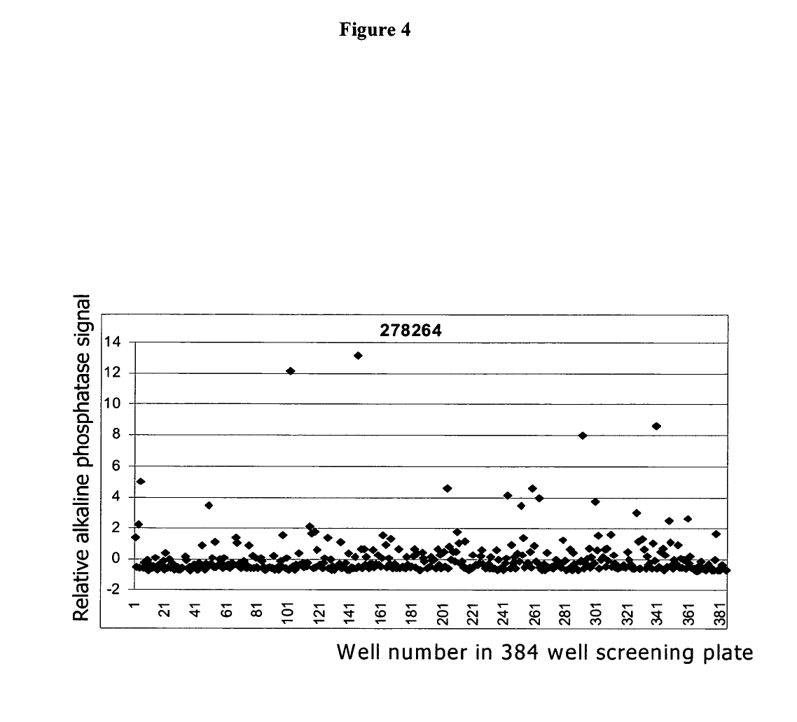

The FLeXSelect viruses, in the presence or absence of matching ligands, are screened according to the protocol described above in duplicate in two independent screens, with each singular sample added on a different plate. If ligands are included in the screening, the protocol is modified: the Ad-cDNA infection is carried out on Day 1, ligands are added on Day 2 and endogenous BAP levels are measured on Day 8. A typical result of a 384 well screening plate is depicted in FIG. 4. Indicated in FIG. 4 are the positions in the 384 well plate on the X-axis and relative alkaline phosphatase signals on the Y-axis. The relative alkaline phosphatase signal for a given sample is calculated as the number of standard deviations above the mean for all data points in a given batch (or experiment).

### EXAMPLE 2

### Target Identification Using the AP Assay

Targets are selected according to the following selection criteria:
1) AP signals higher than the mean plus 3 times the standard deviation of all samples (data points) in the batch. The two individual data points within each batch are analyzed independently.
2) Positive AP signals, as defined by criterion 1, for at least two of the four or 3 of the four virus samples that are screened in duplicate in two independent experiments (total of 4 measurements per virus).

Table 1 lists the targets identified according to the above criteria in the alkaline phosphatase assay.

For some of the targets, agonist ligands are known. These can be used to validate the osteogenic potential of the target genes in MPCs: addition of increasing concentrations of ligand to the medium of MPCs (over-expressing the target protein) should dose-dependently increase the up-regulation of the endogenous alkaline phosphatase activity. This is for example observed when MPCs are infected with Ad-NR1H3 and treated with T0901317, and when MPCs are infected with Ad-GPR65 and treated with 1-b-D-Galactosylsphingosine, and when MPCs are infected with Ad-AVPR2 and treated with [deamino-Cysl,D-Arg8]-Vasopressin.

Ad-NR1H3 and T0901317

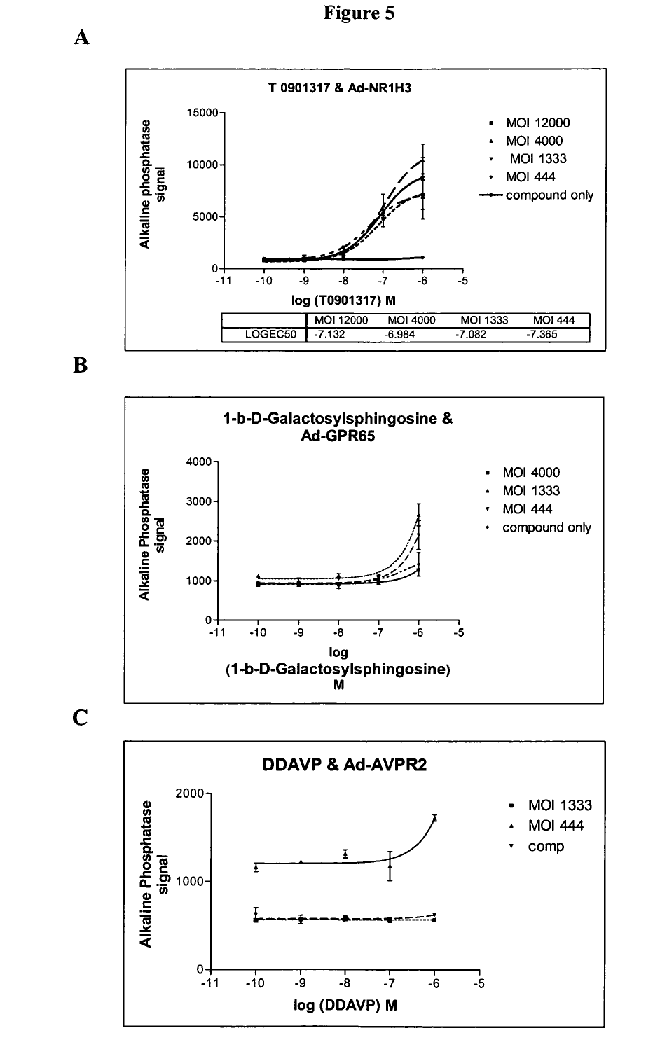

These dose-response curves are depicted in FIG. 5. A dose-response curve for AP activity is generated for MPCs infected with Ad-NR1H3 and treated with T0901317 (FIG. 5A). MPCs are seeded on day 0 at 1000 cells per well of a 384 well plate and co-infected the next day using AdC51-hCAR (MOI 250) and different MOIs of Ad5-NR1H3 (MOI 12000, 4000, 1333, 444). On day 1, 5 concentrations (1E-10M, 1E-9M, 1E-8M, 1E-7M, 1E-6M) of the compound T0901317 (Cayman Chemical, Michigan, USA, Cat. No. 71810) with fixed vehicle concentration (the vehicle is DMSO at the concentration is 0.01%) are added to the wells. After incubation for 6 days at 37° C., 10% CO₂ in a humidified incubator, up-regulation of alkaline phosphatase is read: 15 μl MUP is added to each well, the plates are incubated for 15 min at 37° C. and monitored for AP activity using a fluorescence plate reader (Fluostar, BMG).

Dose-response curves for AP activity are generated in a similar way for MPCs infected with Ad-GPR65 and treated with 1-b-D-Galactosylsphingosine (FIG. 5B); for MPCs infected with Ad-AVPR2 and treated with [deamino-Cysl, D-Arg8]-Vasopressin (DDAVP) (FIG. 5C). Three targets are identified that show a dose-dependent up-regulation of AP activity in the AP assay, when the respective ligands are added at different concentrations.

AdNR1H3 and GW3965

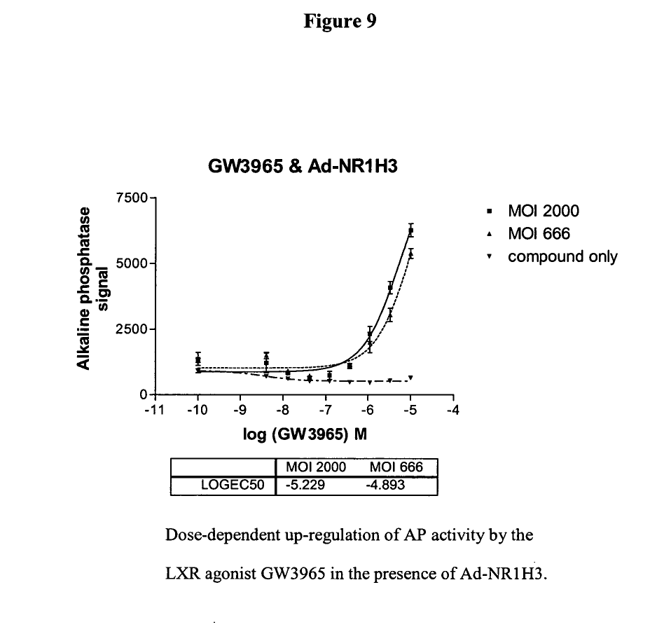

A dose-response relation is observed for AP activity when MPCs are infected with Ad-NR1H3 and treated with GW3965 (FIG. 9). MPCs are seeded on day0 at 1000 cells per well of a 384 well plate and co-infected the next day using AdC51-hCAR (MOI 250) and different MOIs of Ad5-NR1H3 (MOI 2000, 666). On day 1, 8 concentrations (3,43E-9M, 1,34E-8M, 5,35E-8M, 1,60E-7M, 4,81E-7M, 1,43E-6M; 4,29E-6M, 13E-6M) of the compound GW3965 (Chemovation, West Sussex) with fixed vehicle concentration (DMSO at final concentration of 0.1%) are added to the wells. After 6 days, medium is removed and replaced with fresh medium containing the same concentrations of the compound GW3965. Readouts of AP activity are performed at several time points after the start of the experiment, typically after 7, 10 and 13 days. Up-regulation of alkaline phosphatase activity is read as follows: medium is removed from the mono-layers, 15 μl MUP is added to each well, the plates are incubated for 15 min at 37° C. and then read for AP activity using a fluorescence plate reader (Fluostar, BMG). FIG. 9 illustrates the dose-response activity of GW3965 in the presence of Ad-NR1H3.

AdNR1H2 and T0901317

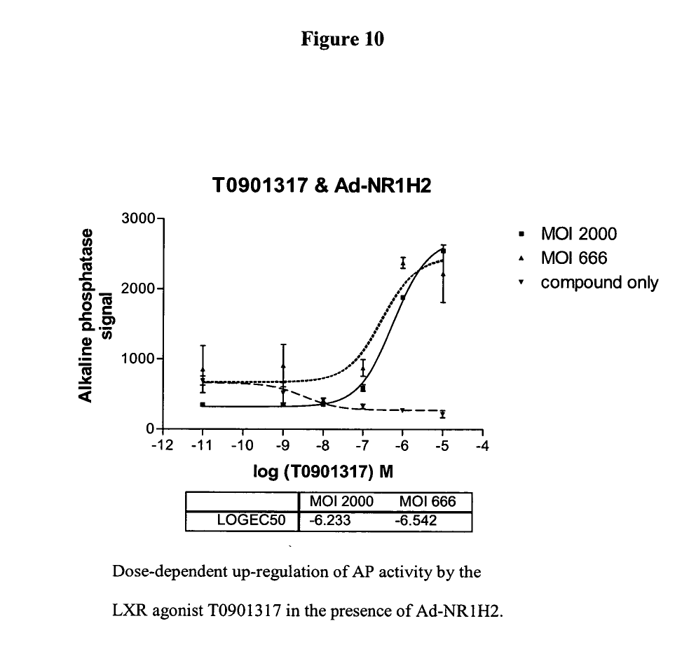

A dose-response relation is observed for AP activity when MPCs are infected with Ad-NR1H2 and treated with T0901317 (FIG. 10). MPCs are seeded on day0 at 1000 cells per well of a 384 well plate and co-infected the next day using AdC51-hCAR (MOI 250) and different MOIs of Ad5-NR1H3 (MOI 2000, 666). On day 1, 5 concentrations (1E-9M, 1E-8M, 1E-7M, 1E-6M, 1E-5M) of the compound T0901317 (Cayman Chemical, Michigan, USA, Cat. No. 71810) with fixed vehicle concentration (DMSO at final concentration of 0.1%) are added to the wells. After 6 days, medium is removed and replaced with fresh medium containing the same concentrations of the compound T0901317. Readouts of AP activity are performed at several time points after the start of the experiment, typically after 7, 10 and 13 days. Up-regulation of alkaline phosphatase activity is read as follows: medium is removed from the monolayers, 15 μl MUP is added to each well, the plates are incubated for 15 min at 37° C. and then read for AP activity using a fluorescence plate reader (Fluostar, BMG). FIG. 10 illustrates the dose-response activity of T0901317 in the presence of Ad-NR1H2.

In conclusion, AP activity is up-regulated in cells transduced with either NR1H3 and NR1H2 in a dose-dependent manner when LXR agonists, GW3965 and T0901317, respectively, are added to the cells at different concentrations in the AP assay.

### EXAMPLE 3

### mRNA and Protein Expression Analysis for the Identified Targets

The assay presented in Example 1 demonstrates the discovery of proteins with osteogenic potential upon overexpression. In order to confirm that these proteins are endogenously expressed in bone forming cells such as MPCs or primary human osteoblasts (hOBs), mRNA is extracted from these cells and expression analyzed using real-time RT-PCR.

Expression levels of target genes are determined in 4 different isolates of MPCs and 2 different isolates of hOBs. The MPCs (obtained from human bone marrow (Cambrex/Biowhittaker, Verviers, Belgium) and hOBs (obtained from Cambrex/Biowhittaker, Verviers, Belgium) are seeded at 3000 resp. 5000 cells/cm² in T180 flasks and cultured until they reached 80% confluency. The cells are washed with ice cold PBS and harvested by adding 1050 μl SV RNA Lysis Buffer to T180 flask. Total RNA is prepared using the SV Total RNA isolation System (Promega, Cat # Z3100). The concentration of the total RNA is measured with the Ribogreen RNA Quantification kit (Molecular Probes, Leiden, The Netherlands, Cat No. R-11490). cDNA synthesis is performed using 40 ng total RNA per reaction using the TaqMan Universal PCR Master Mix, No AmpErase UNG, kit (Applied Biosystems, Warrington, UK, Part number 4324018). For each reverse transcriptase (RT) reaction a minus-RT reaction (negative control: no enzyme included in the reaction) is performed.

The real-time reverse transcriptase (rtRT)-PCR reaction is performed with gene specific primers (Table 2) on both cDNA and minus-RT samples, using the SYBR Green PCR Master Mix (Applied Biosystems, Warrington, UK, Part number 4309155). Primers are quality controlled by performing PCR reactions on human genomic DNA and on plasmids containing the cDNA encoded by the gene studied. If the quality is unsatisfactory, additional primers are designed or validated primer sets are purchased (ABI). For the normalization of the expression levels a RT-PCR reaction is performed on human β-actin using the Human β-actin kit (Applied Biosystems, Warrington, UK, Part number 4310881E). The following program is run on a real-time PCR apparatus (ABI PRISM 7000 Sequence Detection System): 10 min at 25° C., 30 min at 48° C., 5 min at 95° C. Expression levels for the target genes in multiple MPC and hOB isolates are compared to expression levels of β-actin.

TABLE 2

Primers used for the expression analysis of the
target genes.

| | | | |
|---|---|---|---|
| Gene | Primer name | Sequence | SEQ ID |

| | | | |
|---|---|---|---|
| NR1H3 | NR1H3_for #2 | GGGAAGACTTTGCCAAAGCA | 35 |
| NR1H3 | NR1H3_rev #2 | TCGGCATCATTGAGTTGCA | 36 |
| ADORA2A | ADORA2A_for | ATCCCGCTCCGGTACAATG | 39 |
| ADORA2A | ADORA2A_rev | TCCAACCTAGCATGGGAGTCA | 40 |
| RE2/GPR161 | RE2_for | ATTGCCATCGACCGCTACTATG | 43 |
| RE2/GPR161 | RE2_rev | CAGCCGATGAGCGAGTGAA | 44 |
| HSU93553 | HSU93553_for | CCGACAAGTGGTACATGGAAAG | 45 |
| HSU93553 | HSU93553_rev | CTCCGGCTTGTGATGCTATTATG | 46 |
| GPR52 | GPR52_for | TGCGTCCGAGCGTCACT | 49 |
| GPR52 | GPR52_rev | ATGCAGACATCCACCACACTGT | 50 |
| MC5R | MC5R_For | TCCGTGATGGACCCTCTCATATAT | 51 |
| MC5R | MC5R_rev | GGCAGCAAATAATCTCCTTAAAGGT | 52 |
| GPR65 | GPR65_for | CTTTGGTCACCATCCTGATCTG | 53 |
| GPR65 | GPR65_rev | TTCCTTGTTTTCCGTGGCTTTAT | 54 |
| GPR12 | GPR12_for | GCTGCCTCGGGATTATTTAGATG | 55 |
| GPR12 | GPR12_rev | TCTGGCTCTACGGCAGGAA | 56 |
| AVPR2 | AVPR2_for | TGTGAGGATGACGCTAGTGATTG | 57 |
| AVPR2 | AVPR2_rev | CAGCAACATGAGTAGCACAAAGG | 58 |
| DRD1 | DRD1_for | GTAACATCTGGGTGGCCTTTG | 59 |
| DRD1 | DRD1_rev | ACCTGTCCACGCTGATCACA | 60 |
| ESRRG | ESRRG_for | AAAGTGGGCATGCTGAAAGAA | 61 |
| ESRRG | ESRRG_rev | CGCATCTATCCTGCGCTTGT | 62 |

### EXAMPLE 4

### Analysis of the Up-Regulation of Endogenous Bone AP mRNA Versus that of Placental or Intestinal AP mRNA

Bone alkaline phosphatase (BAP) is the physiologically relevant alkaline phosphatase (AP) involved in bone formation. In order to determine whether the measured AP activities are due to up-regulation of BAP expression or of another AP, mRNA levels for all AP genes are analyzed after infection of MPCs.

mRNA levels are determined as described in the previous section. The difference is in the primer set used (Table 3): one set detects BAP ALPL (human alkaline phosphatase liver/bone/kidney) mRNA expression. Another set detects the expression of the 3 other AP genes (ALPI (human alkaline phosphatase intestinal), ALPP (human alkaline phosphatase placental (PLAP)), ALPPL2 (human alkaline phosphatase placental-like)). ALPI, ALPP and ALPPL2 are highly similar at the nucleotide level and can therefore be amplified using one primer pair.

TABLE 3

Primer sets used to analyze mRNA expression of
different alkaline phosphatase isoforms.

| | | |
|---|---|---|
| Name | sequence | SEQ ID NO: |

| | | |
|---|---|---|
| JDO-05F (PLAP) | TTCCAGACCATTGGCTTGAGT | 65 |
| JDO-05bis R | ACTCCCACTGACTTTCCTGCT | 66 |
| (PLAP/ALPI/ALPPL2) | | |
| JDO-21F (BAP) | CATGCTGAGTGACACAGACAAGAAG | 67 |
| JDO-21R (BAP) | TGGTAGTTGTTGTGAGCATAGTCCA | 68 |

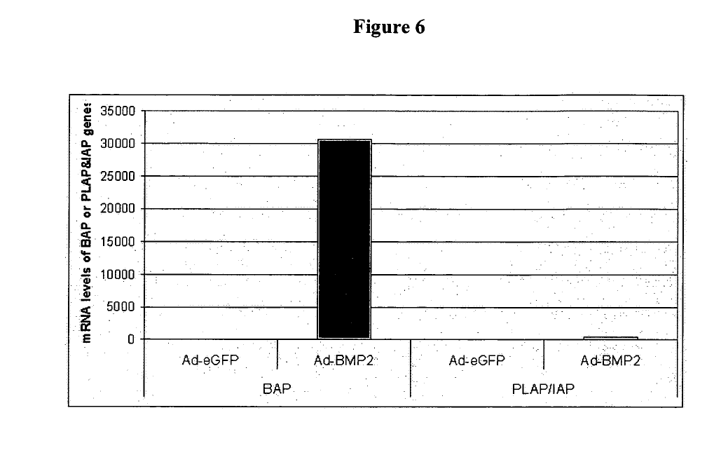

The primer pairs are first validated on RNA isolated from MPCs infected with Ad-eGFP and Ad-BMP2. FIG. 6 illustrates the strong up-regulation of BAP mRNA by Ad-BMP2 and the absence of up-regulation of expression of any of the other AP genes. Both primer sets are then used to measure mRNA levels for all AP genes in RNA isolated from Ad-target infected MPCs.

### EXAMPLE 5

### Analysis of Expression Levels of NR5A2, NR1H3, NR1H2, ESRRG in Cell Types Relevant to Bone Formation

To confirm that the identified target genes are endogenously expressed in cell types that relate to bone formation, mRNA levels for these genes are determined in relevant cell types.

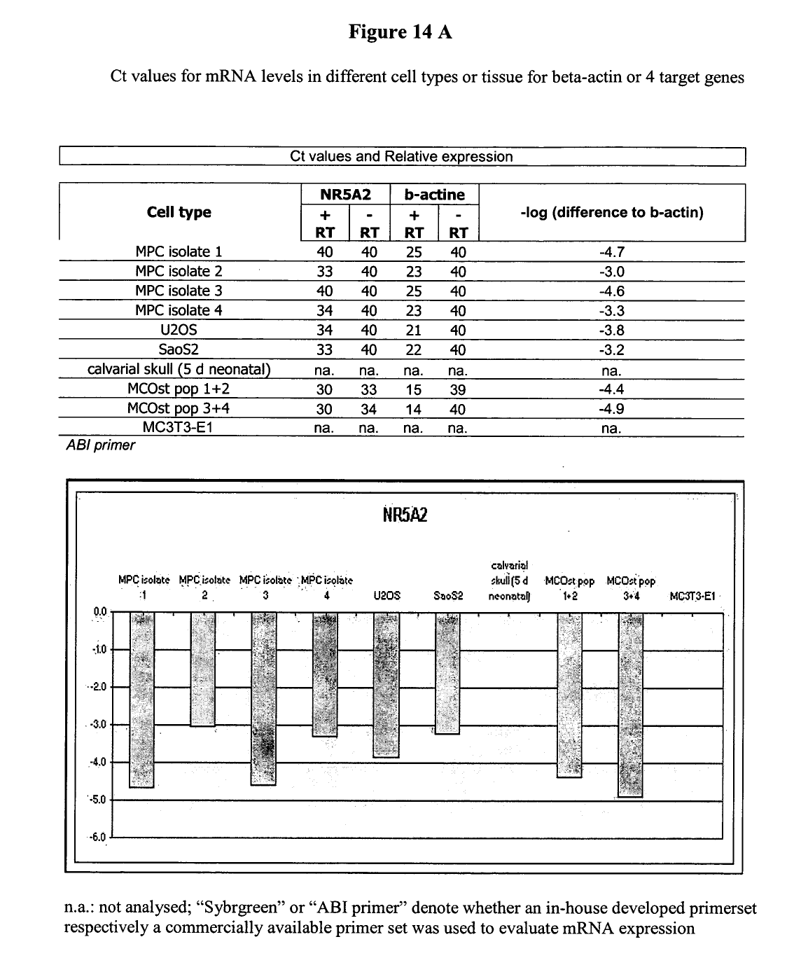

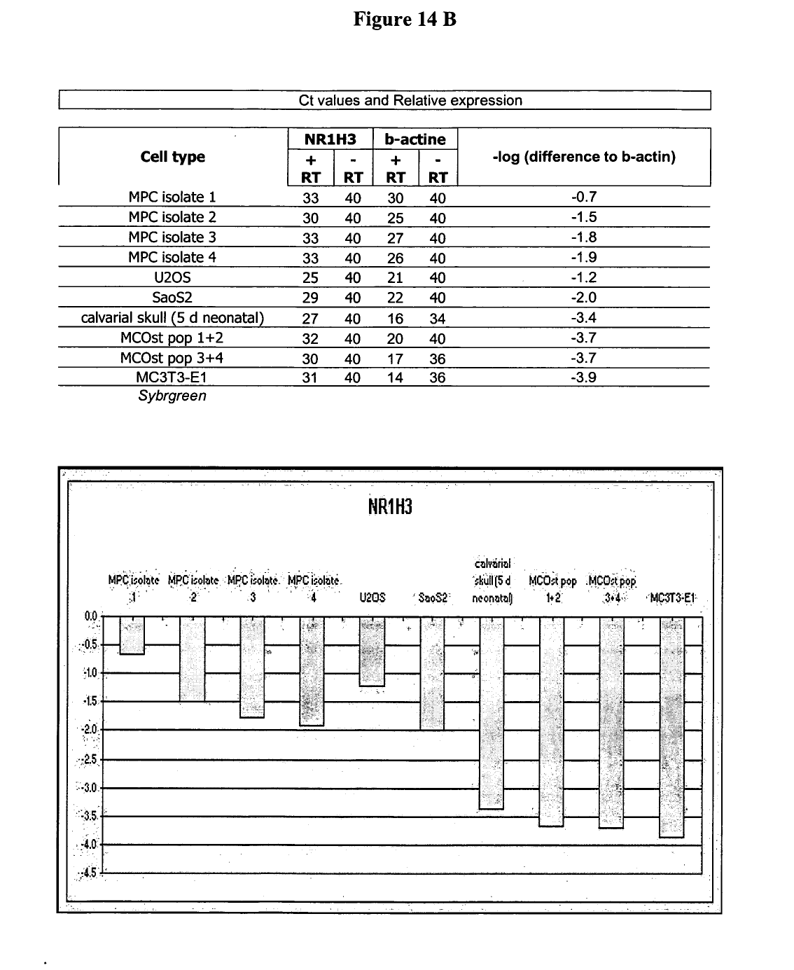

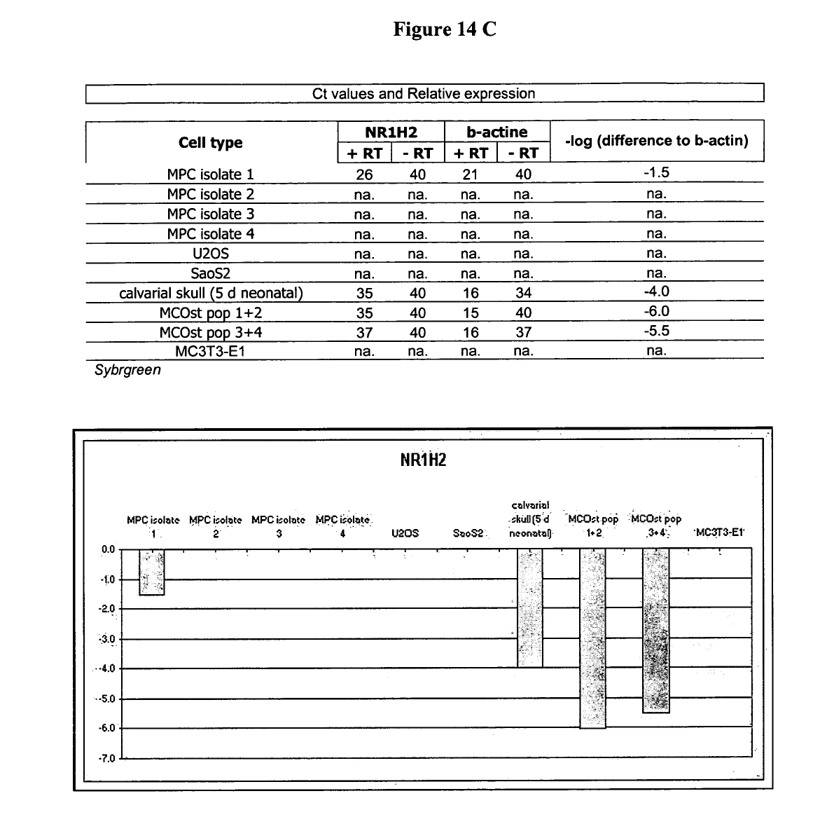

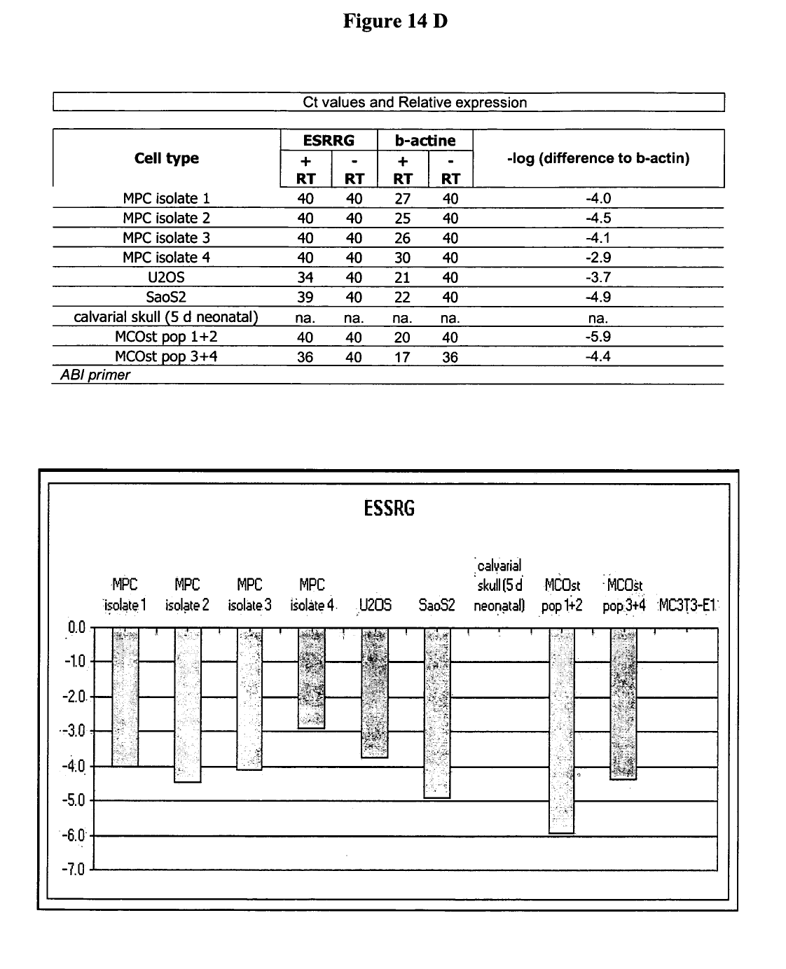

Primary cells or cell lines (FIG. 14A-D: MPC isolates 1-4, calvarial osteoblasts (MCOst pop 1+2, 3+4)), human osteoblast cell lines (SaOS2, U20S) are cultured or calvarial skull tissue is harvested from 5-day old mice. Monolayers or skull tissue is harvested and total RNA is extracted (SV Total RNA isolation. System, Promega # Z3100) and quantified (Ribogreen RNA Quantification kit, Molecular Probes, Leiden). cDNA synthesis is performed using 20 ng total RNA per reaction using the TaqMan Universal PCR Master Mix, No AmpErase UNG, kit (Applied Biosystems, Warrington, UK, Part number 4324018). For each reverse transcriptase (RT) reaction a minus-RT reaction (negative control: no enzyme included in the reaction) is performed. The real-time reverse transcriptase (rtRT)-PCR reaction is performed with gene specific primers on both cDNA and minus-RT samples, using the SYBR Green PCR Master Mix (Applied Biosystems, Warrington, UK, Part number 4309155). Primers are quality controlled by performing PCR reactions on human genomic DNA and on plasmids containing the cDNA encoded by the gene studied if available. If the quality is unsatisfactory, additional primers are designed or validated, and primer sets are purchased (ABI). For the normalization of the expression levels a RT-PCR reaction is performed on human β-actin using the Human β-actin kit (Applied Biosystems, Warrington, UK, Part number 4310881E). The following program is run on a real-time PCR apparatus (ABI PRISM 7000 Sequence Detection System): 10 min at 25° C., 30 min at 48° C., 5 min at 95° C.

Expression levels for the four genes are compared to expression levels of beta-actin and the results shown in FIG. 14 A-D. The figures show the Ct values obtained for analysing mRNA levels in different cell types or tissue for beta-actin or 4 target genes; n.a.: not analysed; “Sybrgreen” or “ABI primer” denote whether an in-house developed primerset respectively a commercially available primerset was used to evaluate mRNA expression. Also shown are the graphic representation of the differential expression levels of target genes versus beta-actin expression levels (values are taken from left columns from the data tables).

In conclusion, the identified target genes are expressed in multiple cell types relevant to bone formation. It should be noted that target gene ESRRG is not expressed in the MPC isolates tested.

### EXAMPLE 6

### Activity of LXR Agonists in the BAP Assay, Upon Over-Expression of NR1H2 or NR1H3

Ad-NR1H2 and GW3965

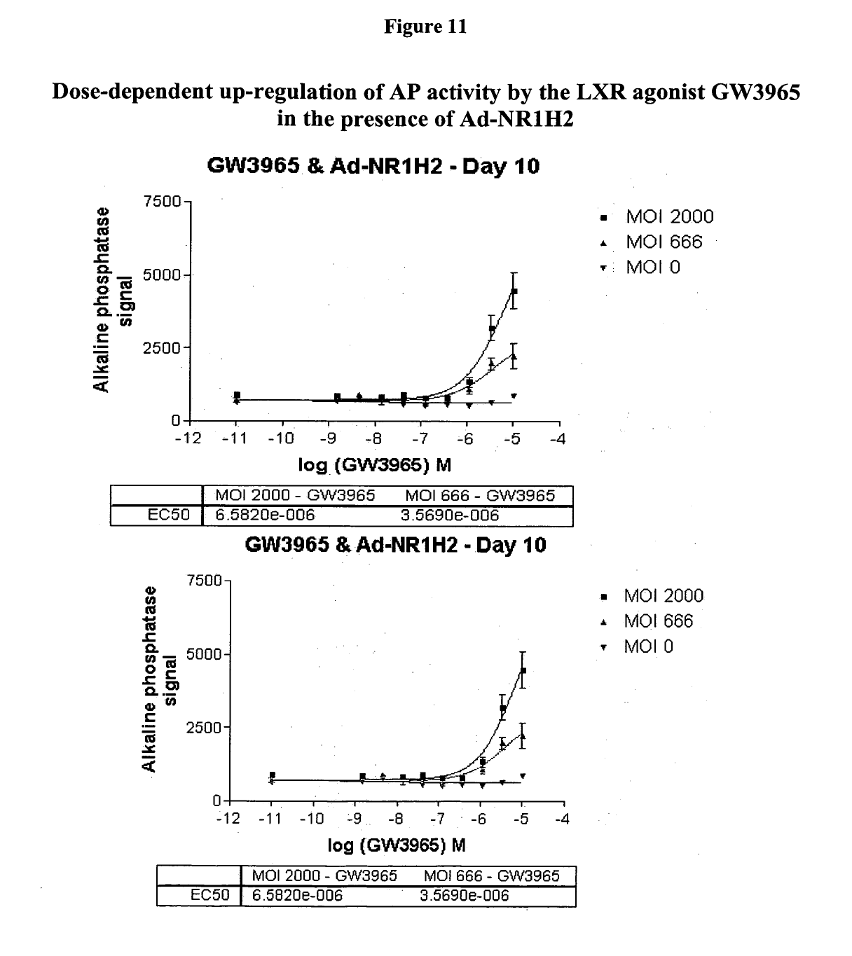

A dose-response relation is observed for AP activity when MPCs are infected with Ad-NR1H2 and treated with GW3965 (FIG. 11). MPCs are seeded on day 0 at 1000 cells per well of a 384 well plate and co-infected the next day using AdC51-hCAR (MOI 250) and different MOIs of Ad5-NR1H2 (MOI 2000, 666). On day 1, 9 concentrations (1.52E-9M, 4.57E-9M, 1.37E-8M, 4.12E-8M, 1.23E-7M, 3.7E-7M, 1.11E-6M, 3.33E-6M, 1E-5M) of the compound GW3965 with fixed vehicle concentration (DMSO at final concentration of 0.161%) are added to the wells. After 6 days, medium is removed and replaced with fresh medium containing the same concentrations of the compound GW3965. Readouts of AP activity are performed at several time points after the start of the experiment, typically after 7, 10 and 13 days. Up-regulation of alkaline phosphatase activity is read as follows: medium is removed from the monolayers, 15 μL MUP is added to each well, the plates are incubated for 15 min at 37° C. and then read for AP activity using a fluorescence plate reader (Fluostar, BMG). FIG. 11 illustrates the dose-response activity of GW3965 in the presence of Ad-NR1H2.

Ad-NR1H2 Ad-NR1H3 and acetyl-podocarpic Dimer (APD)

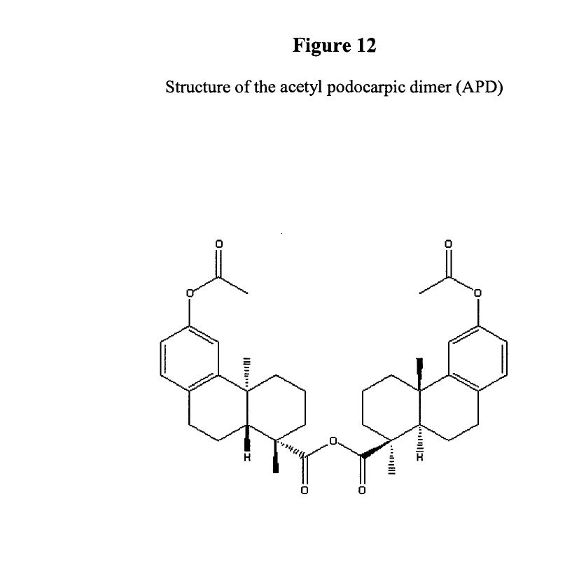

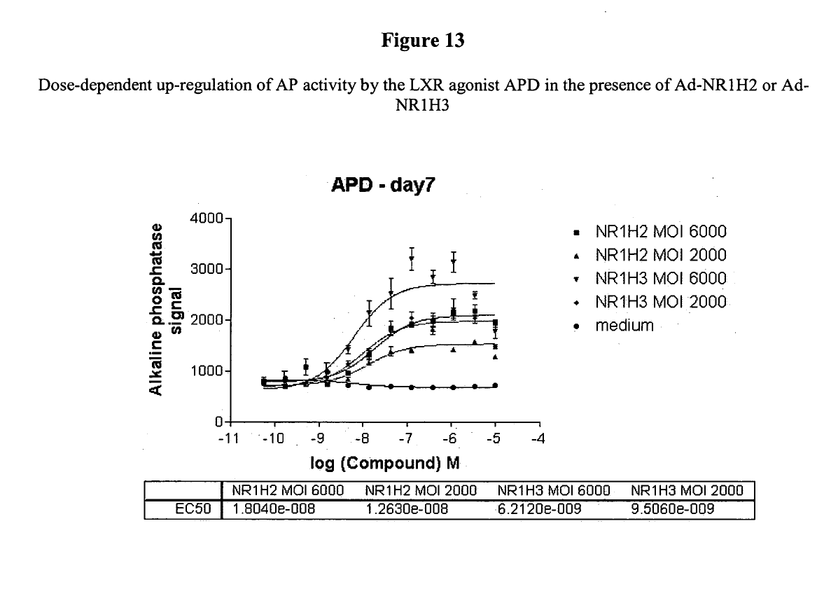

A dose-response relation is observed for AP activity when MPCs are infected with Ad-NR1H2 or Ad-NR1H3 and treated with acetyl podocarpic dimer (APD—see FIG. 12 for compound structure; APD is disclosed as “Compound 1” in published UA2003/0086923A1, of which the preparation of APD is incorporated by reference). MPCs are seeded on day0 at 1000 cells per well of a 384 well plate and co-infected the next day using AdC5′-hCAR (MOI 250) and different MOIs of Ad5-NR1H2 or Ad-NR1H3 (MOI 2000, 6000). On day 1, 12 concentrations (5.65E-11M, 1.69E-10M, 5.08E-10M, 1.52E-9M, 4.57E-9M, 1.37E-8M, 4.12E-8M, 1.23E-7M, 3.7E-7M, 1.1 E-6M, 3.33E-6M, 1E-5M) of the compound APD with fixed vehicle concentration (DMSO at final concentration of 0.1%) are added to the wells. After 6 days, medium is removed and replaced with fresh medium containing the same concentrations of the compound APD. Readouts of AP activity are performed at several time points after the start of the experiment, typically after 7, 10 and 13 days. Up-regulation of alkaline phosphatase activity is read as follows: medium is removed from the monolayers, 15 μl MUP is added to each well, the plates are incubated for 15 min at 37° C. and then read for AP activity using a fluorescence plate reader (Fluostar, BMG). FIG. 13 illustrates the dose-response activity of APD in the presence of Ad-NR1H2 or Ad-NR1H3.

In conclusion, AP activity is up-regulated in cells transduced with either NR1H3 or NR1H2 in a dose-dependent manner when LXR agonists, APD, GW3965 and T0901317, respectively, are added to the cells at different concentrations in the AP assay.

### EXAMPLE 7

### Osteogenic Pathway Analysis: NR5A2 and NR1H3+T0901317 Up-Regulate mRNA Levels of Osteogenic Markers

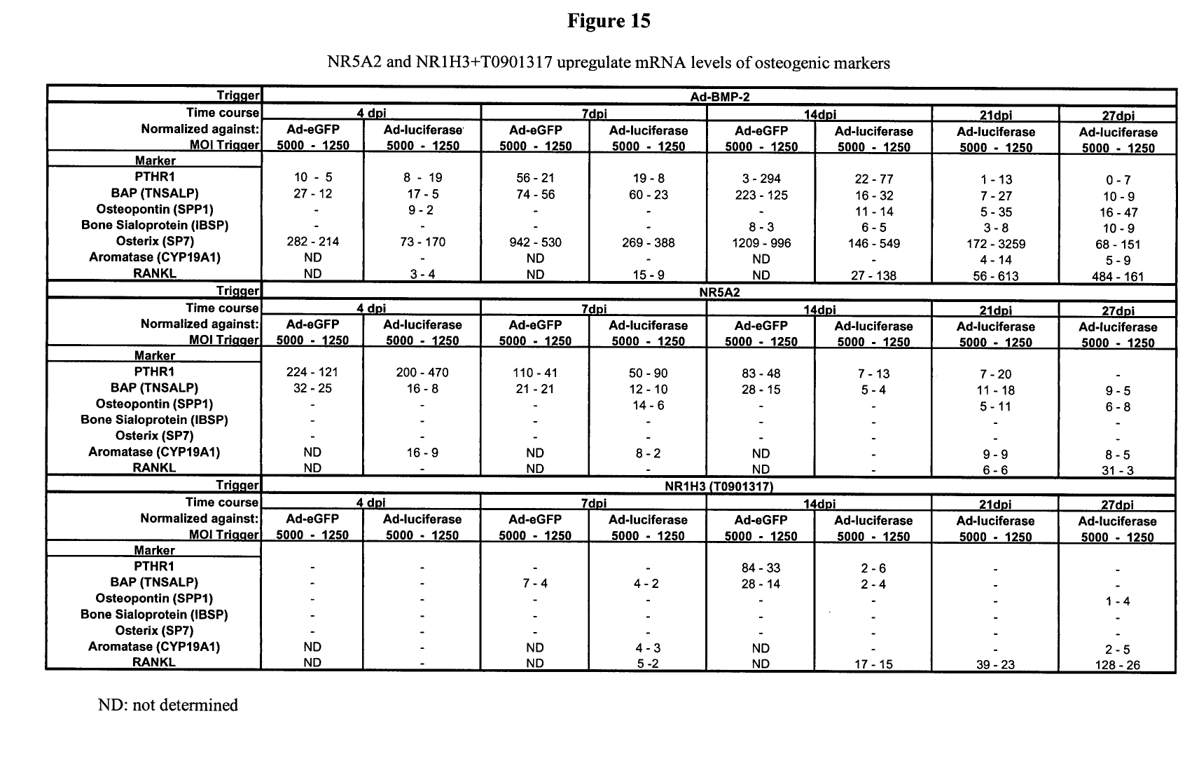

Osteogenic differentiation of MPCs into osteoblasts is accompanied by the up-regulation of osteogenic proteins. The latter are useful to study the induction of osteogenic differentiation by a novel target using for example real-time RT-PCR. The MPCs that are used in this study are profiled for the up-regulation of a limited set of osteogenic markers by BMP2. Markers that show differential expression for BMP2 are subsequently tested against mRNA derived from Ad-NR5A2 infected cells or derived from Ad-NR1H3+T0901317 treated cells. 100,000 MPCs are seeded in each well of a 6 well plate in 2 ml MPC medium, containing 10% FCS. The next day, after incubation at 37° C., 10% CO₂ in a humidified incubator, cells are co-infected with AdC15-hCAR (final MOI of 750) and Ad-NR5A2, Ad-NR1H3+T0901317 (1 μM) or Ad-BMP2 (positive control) or Ad-eGFP or Ad-luciferase as negative controls (final MOIs of 1250 and 2500). Cells are incubated at 37° C., 10% CO₂ in a humidified incubator for a further six days unless cells are already harvested for RNA isolation. Virus is removed and replaced by 2 ml fresh OS medium (proprietary medium containing 10% FCS). Over the next 3 weeks, medium is refreshed 3 times per 2 weeks. Every other time, medium is refreshed half or completely. Monolayers are harvested at several time points (see FIG. 15), total RNA is harvested and quantified and rtRT-PCRs are run as follows: monolayers are washed with ice cold PBS and harvested by adding SV RNA Lysis Buffer. Total RNA is prepared using the SV Total RNA isolation System (Promega, Cat # Z3100). RNA concentration is measured with the Ribogreen RNA Quantification kit (Molecular Probes, Leiden, The Netherlands, Cat No. R-11490). cDNA synthesis is performed using 20 ng total RNA per reaction using the TaqMan Universal PCR Master Mix, No AmpErase UNG, kit (Applied Biosystems, Warrington, UK, Part number 4324018). For each reverse transcriptase (RT) reaction a minus-RT reaction (negative control: no enzyme included in the reaction) is performed. The real-time reverse transcriptase (rtRT)-PCR reaction is performed with gene specific primers on both cDNA and minus-RT samples, using the SYBR Green PCR Master Mix (Applied Biosystems, Warrington, UK, Part number 4309155). Primers are quality controlled by performing PCR reactions on human genomic DNA and on plasmids containing the cDNA encoded by the gene studied if available. If the quality is unsatisfactory, additional primers are designed or validated primer sets are purchased (ABI). For the normalization of the expression levels a RT-PCR reaction is performed on human β-actin using the Human β-actin kit (Applied Biosystems, Warrington, UK, Part number 4310881E). The following program is run on a real-time PCR apparatus (ABI PRISM 7000 Sequence Detection System): 10 min at 25° C., 30 min at 48° C., 5 min at 95° C.

Expression levels for osteogenic marker genes are first normalized for beta-actin levels. The resulting data for Ad-BMP2, Ad-NR5A2 and Ad-NR1H3+T0901317 (1 μM) samples are then compared to those of Ad-eGFP or Ad-luciferase negative control samples, harvested at the same time points, for cells infected at the same MOI. The fold up-regulation of marker gene mRNA induced by NR5A2 or BMP2 over-expression are calculated and presented in FIG. 15. Osteogenic markers are considered to be up-regulated by BMP2, NR5A2 or NR1H3+T0901317 over-expression if their expression is 4-fold higher than that in a negative control sample (Ad-eGFP or Ad-luciferase). Ad-NR5A2 up-regulated expression of PTHR1, BAP, osteopontin, aromatase and RANKL at one or more time points studied. Ad-NR1H3+T0901317 up-regulated expression of PTHR1, BAP, osteopontin, aromatase and RANKL at one or more time points studied.

### EXAMPLE 8

### Osteogenic Pathway Analysis: Up-Regulation of NR5A2 and NR1H3 mRNA Levels by Osteogenic Triggers

MPCs are treated with established inducers of osteogenesis and NR5A2 or NR1H3 mRNA levels are determined in an effort to place NR5A2 or NR1H3 in known osteogenic pathways.

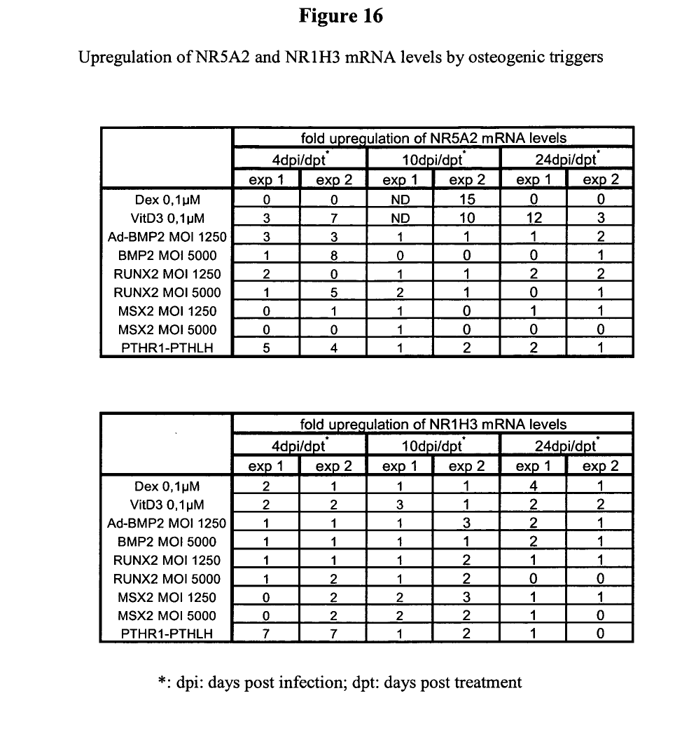

100,000 MPCs are seeded in each well of a 6 well plate in 2 ml MPC medium, containing 10% FCS. The next day, after incubation at 37° C., 10% CO₂ in a humidified incubator, cells are co-infected with AdC15-hCAR (final MOI of 750) and Ad-BMP2, Ad-RUNX2, Ad-MSX2, Ad-PTHR1/PTHLH or Ad-eGFP or Ad-luciferase as negative controls (final MOIs of 1250 and 2500). Alternatively, cells are treated with dexamethasone (final concentration 0.1 μM), VitD3 (final concentration 0.1 μM) or the vehicle controls (0.1% EtOH or DMSO). Cells are incubated at 37° C., 10% CO₂ in a humidified incubator for a further six days unless cells are already harvested for RNA isolation. Virus is removed and replaced by 2 ml fresh OS medium (proprietary medium containing 10% FCS). Over the next 18 days, medium is refreshed 3 times per 2 weeks. Every other time, medium is refreshed half or completely. Monolayers are harvested at several time points (see FIG. 16), total RNA is harvested and quantified and rtRT-PCRs is run as described in the previous example “NR5A2 and NR1H3+T0901317 up-regulate mRNA levels of osteogenic markers”. The fold up-regulation of NR5A2 or NR1H3 mRNA compared to negative controls (vehicle for dexamethasone or VitD3 treatment) or Ad-luciferase for Ad-infections) is calculated (FIG. 16).

NR5A2 mRNA levels became up-regulated by VitD3 treatment at several time points and NR1H3 and NR5A2 levels by Ad-PTHR1/PTHLH infection at the 4 dpi time point.

### EXAMPLE 9

### Mineralization Assay

The process of osteogenesis consists of several successive events. During the initial phases of osteogenesis, BAP becomes up-regulated, while mineralization is a specific event occurring in later stages of osteogenesis.

Bone tissue consists of cells embedded in a matrix of organic materials (e.g., collagen) and inorganic materials (Ca²⁺ and phosphate). Bone mineralization is shown in vitro by staining differentiated bone cells for the matrix they deposited. The Von Kossa and Alizarin RedS stains allow the visualization of deposited phosphate and calcium, respectively.

On day one, primary human MPCs are seeded in a 6 well plate (Costar or Nunc) at a density of 50,000 to 250,000 cells per well, typically at 100,000 cells per well. MPCs are co-infected one day later with AdC15-hCAR (MOI 750) and Ad-control (eGFP or BMP2) or hit-virus (Ad5) (at MOIs between 250 and 20,000, typically at MOIs 5000 and 2500). For Ad-GPCR or Ad-NHR experiments, cells can additionally be treated with specific ligands. These are added at the EC₅₀ concentration and at concentrations 5-10 times higher and lower. Ligands are added 2-3 times per week. Medium supplemented with 100 μg/ml L-ascorbate and 10 mM beta-glycerophosphate, is refreshed 2 times a week. 20 to 30 days after the start of the experiment, cells are stained with Von Kossa stain or with Alizarin RedS stain.

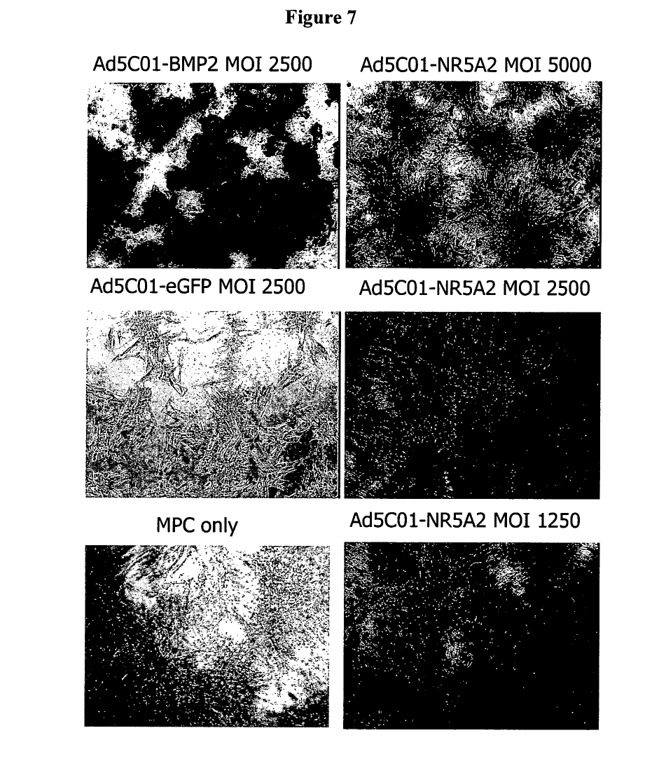

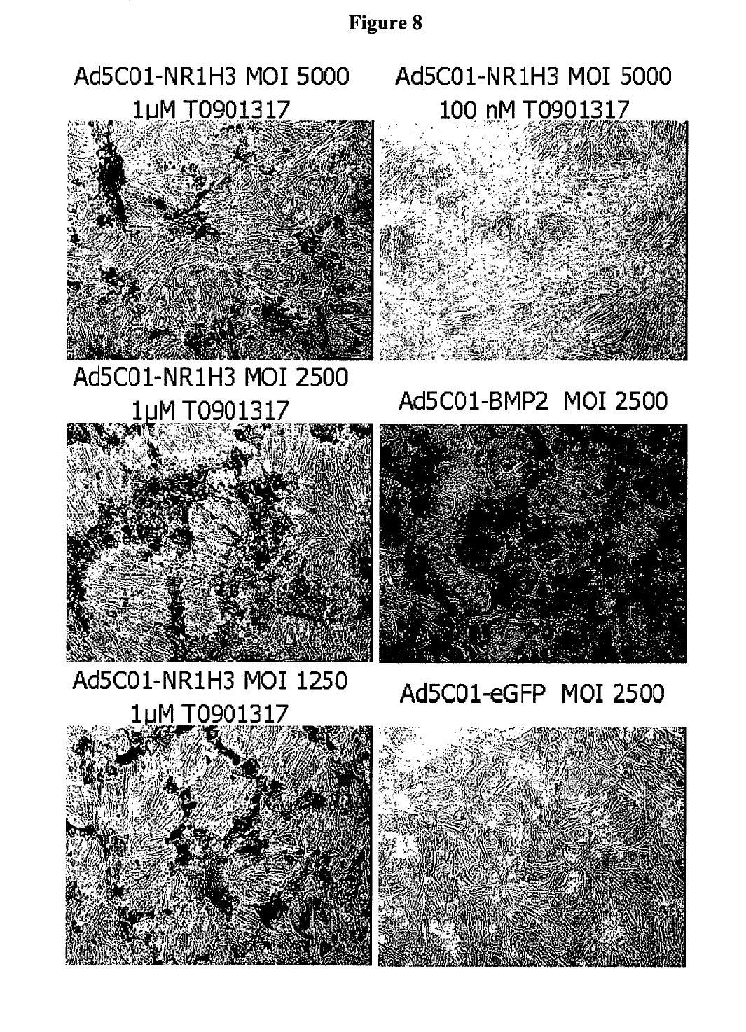

The Alizarin RedS staining is carried out as follows: cells are washed once with PBS, fixed with 10% paraformaldehyde for 45 minutes at 4° C., and washed 2 times with PBS. Cells are incubated with 40 mM aqueous Alizarin RedS solution, pH 4.1-4.3 for 10 minutes followed by 5 washes with distilled water. Staining is evaluated and photographed using white light. Examples are shown in FIGS. 7 and 8.

In conclusion, two targets are already identified that induced mineralization, in the presence or absence of their respective ligands: NR5A2 (FIG. 7) and NR1H3 (FIG. 8).

In studies conducted with calvarial skull tissue, the administering of LXR agonists alone induce bone formation, thereby showing that LXR agonists are useful in the methods of the present invention, including methods for differentiating precursor cells into osteoblasts, for stimulating bone tissue formation, and treating or preventing bone diseases, including treating or preventing osteoporosis.

The data presented in FIGS. 9 and 10 indicate that LXR agonists do not induce the same level of alkaline phosphatase activity in the absence of Ad-NR1H3 or Ad-NR1H2, as in the presence of Ad-NR1H3 or Ad-NR1H2. This finding, which appears inconsistent with the calvarial skull tissue findings, may be the result of many factors, such as, for example, the overexpression of NR1H3 or NR1H2 protein may recruit a different set of coactivator proteins than endogenous NR1H3 or NR1H2 proteins.

### EXAMPLE 10

### Calvarial Skull Assay: Activity of the NR1H3 Agonist T0901317

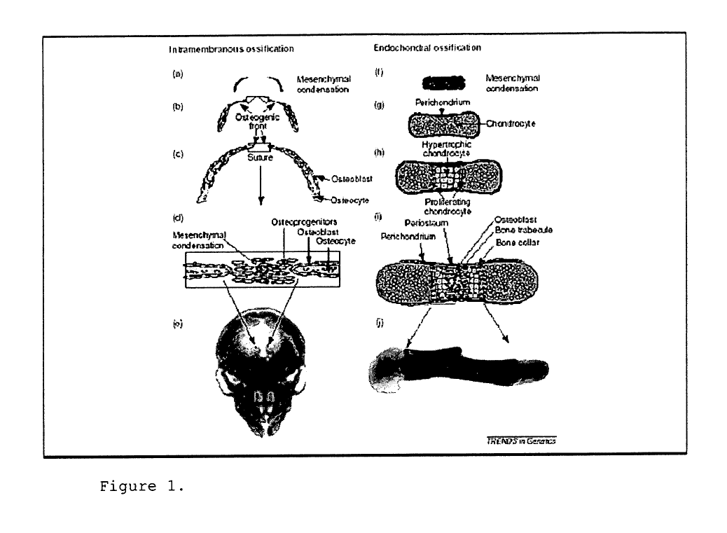

Adult bone consists of organic (e.g. collagen type I) and inorganic (calcium phosphate) material, bone-forming cell types (MPCs, osteoblasts and osteocytes) and bone-degrading cell types (osteoclasts). Since the MPC monolayers, used in the identification and initial validation of the target hits, do not mimic the multi-cellular 3-dimensional in vivo environment, bone organ culture models were developed. Elegant ex vivo models that closely mimic the in vivo bone environment are bone organ cultures, such as the metatarsal or calvarial skull organ culture models. In the former model, foot bones formed by endochondral ossification are used. In the latter model, skull bones, formed by intramembranous ossification are used (see also FIG. 1). This example describes the latter model using calvarial skull bones.

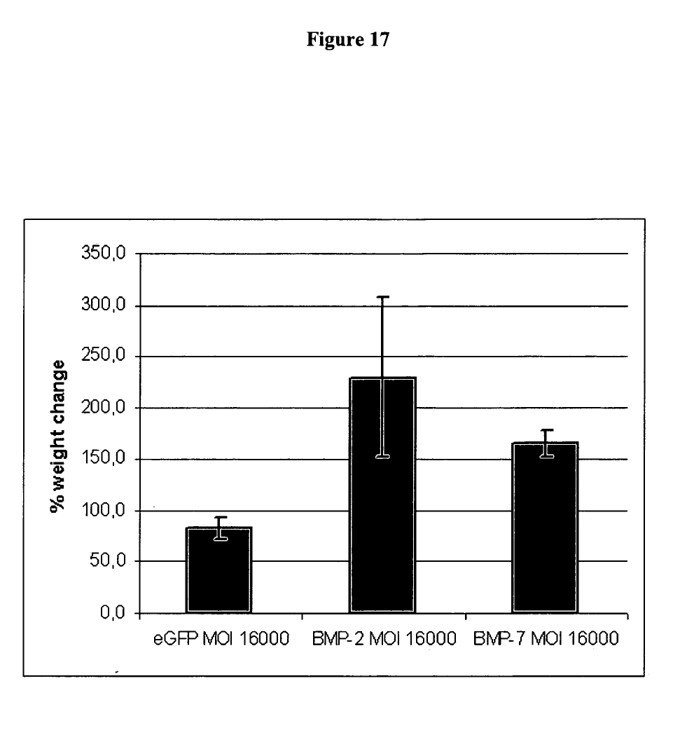

CD1 pups are harvested around birth from CD1 female mice (received from Janvier (Le Genest St Isle, France) when they were 11 days pregnant). Pups are decapitated and the calvarial skull is dissected and split into 2 hemicalvaria. Hemicalvaria are blotted using sterile gauze, weighed and cultured in 24 well plates (MEMalpha or BGJb-Fitton-Jackson medium containing 50 μg/ml L-ascorbic acid (Sigma, A-4034), 5 mM β-glycerophosphate (Sigma, G-9891) and Penicillin-Streptomycin (Invitrogen Cat # 15140-122)). Small molecules (ligands, agonists, antagonists) are tested in at least three-fold at a minimum of 3 concentrations. Each small molecule is added to the medium on day 0 and added again when refreshing the medium (every 2-3 days). Three to 16 days after the start of the experiment, skulls are weighed again after blotting them dry using sterile gauze. The weight difference is calculated, expressed as percent weight change and the mean and standard deviations (SD) are calculated for the triplicate measurements. Data are analyzed using the Student's t-test. Weight increases for Ad-BMP2 and Ad-BMP7 positive controls are depicted in FIG. 17.

The formation of new osteoid is analyzed histologically as follows: hemicalvaria are fixed in 10% buffered formalin for at least 2 days, decalcified in 10% EDTA overnight, processed through graded alcohols and embedded in paraffin wax. Three to 10 μm sections are prepared of the calvaria and stained with hematoxylin and eosin (H&E). Healthy cells, dead cells, old and new bone, and collagen are identified by their distinctive morphology and colouring observed after H&E staining. The surfaces taken by these are measured stereologically (μm² readout) and termed Osteoblast area, Debris area, Native and New bone area, Collagen area and Total area (sum of the previous 5 areas), respectively. In addition, the thickness (μm readout) is measured at 8 positions, evenly spaced over the section.

The histological readout of the calvarial skull assay is developed using known osteogenic agents. Hemicalvaria were treated with recombinant human parathyroid hormone (rhPTH). PTH has a dual action on bone: PTH needs to be administered in vivo intermittently rather than continuously since the latter treatment regimen results in bone resorption, while the former results in bone build-up. This dual action is also observed in the calvarial skull model as expected: PTH at 10⁻⁷ M has a resorptive effect on bone tissue but induces bone build-up at 10⁻¹¹ M.

Since NR1H3 and T0901317 score well in the AP and mineralization assay, the commercially available NR1H3 agonist, T0901317, is tested in the calvarial skull model to further show the osteogenic potential of NR1H3 agonism.

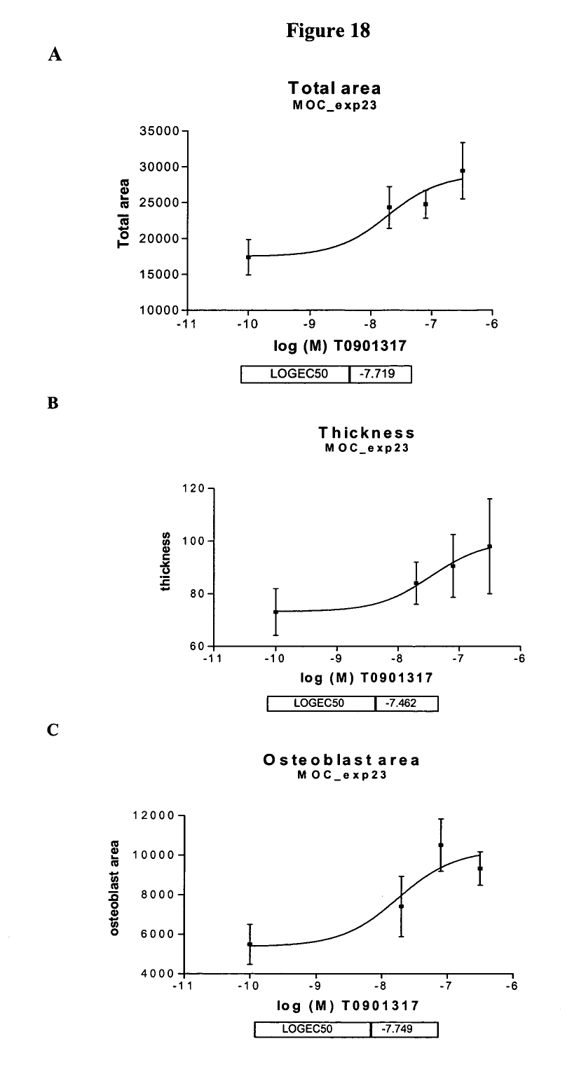

T0901317 is added to the culture medium of the dissected hemicalvaria at the day of dissection at several doses (19.5, 78.1 and 313 nM), in fourfold. The concentration of the solvent (vehicle), DMSO, is fixed at a final concentration of 0.05%. The medium, containing T0901317 or vehicle control is refreshed every 2-3 days. Hemicalvaria are harvested 7 days after the initiation of the experiment and subjected to the histological analysis described above. Statistically significant increases are observed for areas of osteoblast, collagen and new bone. Dose-response activity of the compound is observed towards areas of osteoblast, total area (sum of all areas measured) and thickness (FIG. 18).

Apart from the H&E stainings, other stainings are routinely done. In one method, AP activity is visualized as follows: slides are fixed for 10 min using 4% paraformaldehyde and washed with PBS and MilliQ water. Slides are incubated for 5 min with ALP buffer (ALP buffer: 0.1M Tris-HCl pH 9.5, 20 mM MgCl₂, 100 mM NaCl), blotted using tissue and incubated with substrate (NBT/BCIP (Nitrobluetetrazolium chloride/5-bromo-4-chloro-3-indolyl phosphate, Roche) in ALP buffer). The staining is stopped by washing with MilliQ water when the color turns from yellow into brown.

### EXAMPLE 11

### Dominant-Negative RUNX2 Mutant Interferes with AP Up-Regulation by NR5A2, NR1H3+T0901317 and ESRRG

RUNX2 is a key osteogenic transcription factor relaying many osteogenic triggers received by MPCs or osteoblasts into the appropriate osteogenic transcriptional output. Knockout studies in mice show that RUNX2 is crucial for the ossification of the skeleton during development (Franceschi RT and Xiao G (2003)).

A useful tool to study RUNX2 biology and the osteogenic signals it relays are RUNX2 mutants. A truncated RUNX2 protein lacking the C-terminal transactivating region, but retaining the N-terminal Runt homology DNA binding domain acts as a dominant-negative RUNX2 (DN-RUNX2) protein. This type of mutant can interfere with RUNX2 activity in vitro and in vivo (Zhang et al., 2000). MPCs express significant levels of RUNX2 mRNA (levels are about 10-fold lower than b-actin mRNA levels).

Since the osteogenic activity of BMP2 is known to work through RUNX2, Ad-BMP2 and Ad-DN-RUNX2 viruses are used to develop the DN-RUNX2 assay. The human full-length RUNX2 cDNA is obtained by RT-PCR from total RNA extracted from MPCs. The 5′ part of the cDNA encoding amino acids 1-214 is obtained by PCR from the cloned RUNX2 cDNA and subcloned in an adenoviral adapter plasmid. The identity of the cloned fragment is verified by sequencing. This plasmid is used to generate an adenoviral stock, as described in WO 9964582.

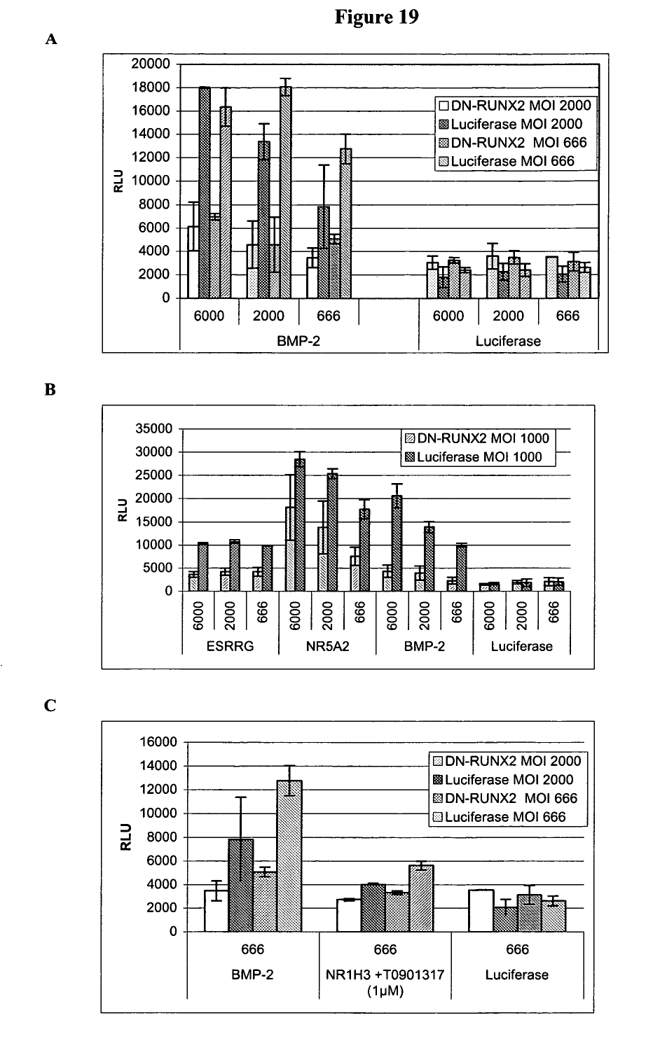

MPCs are seeded at 1000 cells/well in a 384 well plate and infected the next day with adenoviruses encoding hCAR (MOI 250), Ad-BMP2 (MOIs 6000, 2000, 666) and one of Ad-DN-RUNX2 or Ad-luciferase (MOIs 2000 or 666). Alkaline phosphatase activity is read 6 days post infection. From FIG. 19 (A), it is clear that overexpression of DN-RUNX2 significantly reduces the BMP2-induced up-regulation of AP activity. This result shows the functionality of the DN-RUNX2 construct used.

The DN-RUNX2 assay is used to test the involvement of RUNX2 in the up-regulation of AP activity by NR5A2, NR1H3, and ESRRG. MPCs are seeded at 1000 cells/well in a 384 well plate and are infected the next day with adenoviruses encoding hCAR (MOI 250), Ad-BMP2, Ad-ESRRG, Ad-NR5A2, Ad-NR1H3 (MOIs 6000, 2000, 666) and one of Ad-DN-RUNX2 or Ad-luciferase (MOI 1000 or MOIs 2000 and 666) (see FIG. 19 (C)). Alkaline phosphatase activity is read 6 days post infection and raw data are analysed. From FIG. 19 (B), it is clear that overexpression of DN-RUNX2 significantly reduced the ESRRG- and NR5A2-induced up-regulation of AP activity. From FIG. 19 (C), it is clear that overexpression of DN-RUNX2 significantly reduces the up-regulation of AP activity induced by NR1H3 in the presence of T0901317.

### EXAMPLE 12

### Induction of Alkaline Phosphatase Activity by NR5A2, NR1H3+T0901317, ESRRG, Independent of MPC Isolate

MPCs can be isolated, with informed consent, from fresh bone marrow isolated from healthy donors (Cambrex Bioscience/Biowhittaker, Verviers, Belgium). MPCs are a physiologically relevant cell type to isolate osteogenic factors in vitro, using e.g. the AP assay (see Example 2). To exclude targets that function in only one MPC isolate (i.e. from one donor), the targets are also tested on several different MPC isolates to exclude the influence of genetic background in the target discovery process using MPCs.

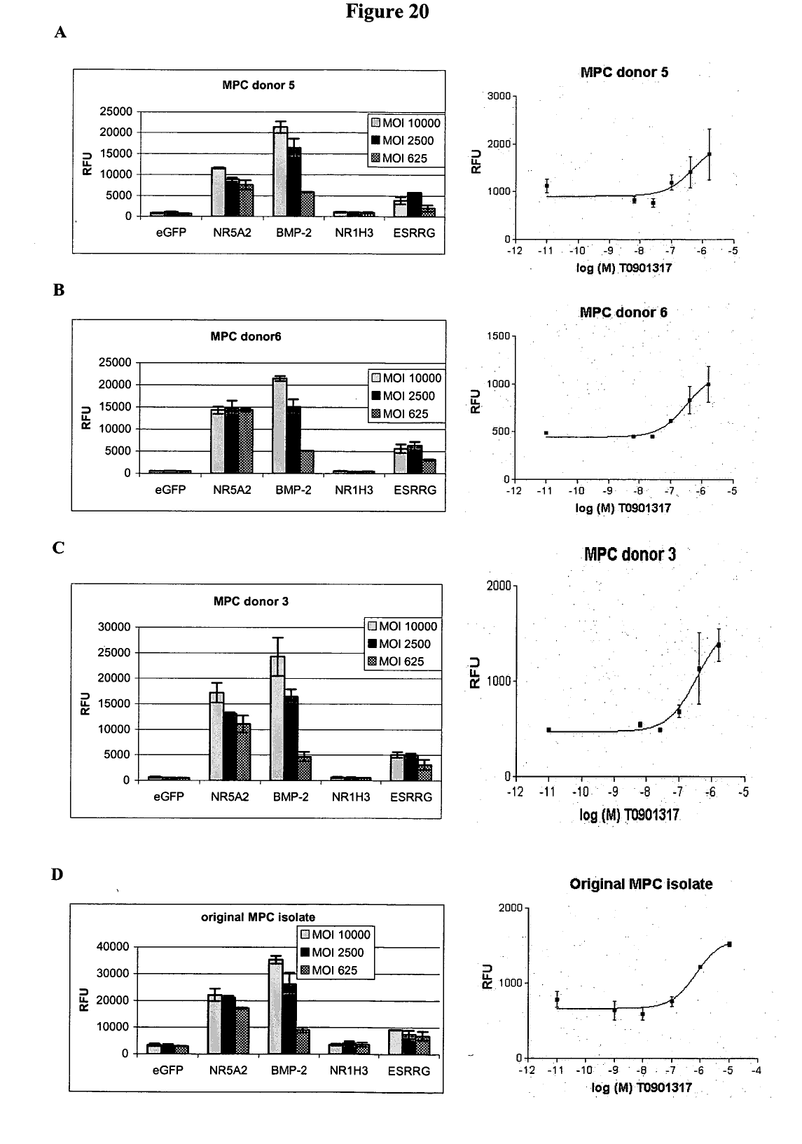

The osteogenic factors NR5A2, NR1H3 and ESRRG are tested in 3 independent MPC isolates different from the one used for target discovery in the AP assay according to a protocol described in Example 2. MPCs are seeded at 1000 cells/well of a 384 well plate and infected the next day with adenoviruses encoding hCAR (MOI 250), Ad-BMP2, Ad-ESRRG, Ad-NR5A2, and Ad-NR1H3 (MOIs 10000, 2500, 625). MPCs infected with Ad-NR1H3 virus at MOI 2500 are also treated one day after infection with T0901317 at different concentrations (FIG. 20) or vehicle. MPCs isolated from 4 different donors (A,B,C,D), are infected with Ad-hCAR, Ad-BMP2 (positive control), Ad-eGFP (negative control), Ad-NR5A2, Ad-ESRRG (data presented in the left panels of A,B,C,D) and Ad-NR1H3+T0901317 (data presented in the right panels of A,B,C,D) together with Ad-luciferase or Ad-DN-RUNX2. 6 days after the start of the infection, endogenous AP activity is measured.

From FIG. 20, it is clear that NR5A2, NR1H3+T0901317 and ESRRG induce AP activity to similar extents in all 4 MPC isolates tested.

### EXAMPLE 13

### Analysis of LXR Agonists for the Treatment of Osteoporosis in the Ovariectomy Animal Model

The gold-standard animal model for analysis of potential osteoporosis therapeutics is the ovariectomy model. Ovariectomy (OVX) results in a drop in estrogen production which is an important causative factor of osteoporosis. This example uses the rat as the animal model, but other animal models such as mice or primates are routinely used by those skilled in the art.

Three-month-old female Lewis rats are maintained under constant conditions of temperature (20±1° C.) and light (12-h light-dark cycle) with ad libitum access to food and water. Rats are sham operated or underwent bilateral ovariectomy after being anesthetized with ketamine and Xylazine. Ovaries are removed after ligation of the uterine horn.

The following groups are formed: sham operated control rats (N=10), ovariectomized rats that receive saline only (OVX, N=12), ovariectomized rats that receive 1713-estradiol (Sigma Chemical Co., St. Louis, Mo., USA) dissolved in small amounts of ethanol with the volume adjusted with olive oil to give a concentration of 30 μg/kg body weight and administered daily subcutaneously for 6 weeks (OVX-E, N=11), ovariectomized rats that receive LXR agonists suspended in the appropriate vehicle (e.g. water and lecithin) and administered daily p.o. for 6 weeks at a dose of 0.1 to 100 mg/kg body weight (OVX-A, N=8). All rats are sacrificed after 6 weeks. On the 2nd, 3^{rd} and 28th day prior to sacrifice, the rats receive xytetracycline (Terramycin, Pfizer) administered intramuscularly at a dose of 20 mg/kg for bone labeling. Femora are obtained for mineralized bone histology and histomorphometry. Bone mineral density (BMD) is measured by dual-energy X-ray absorptiometry (using e.g. apparatus from CTI Concord Microsystems, Knoxyille TN) adapted to the measurement of BMD in small animals. A distal femur scan is performed. In vivo reproducibility is evaluated by measuring the coefficient of variation (CV=100×SD/mean) of five BMD measurements in one rat weighing about 220 g, each time repositioning the rat at the two different sites. The variation is 1.4% in distal femur. In addition, bone alveolar structure is evaluated. All parameters are measured twice, i.e., at baseline and after 6 weeks.

The distal right femur is fixed in 70% ethanol, dehydrated, embedded in methylmethacrylate, and sectioned longitudinally using a Policut S microtome (Reichert-Jung, Heidelberg, Germany). 5- and 10-μm sections are obtained from the center of each specimen. The 5-μm section is stained with 0.1% toluidine blue, pH 6.4, and at least two non-consecutive sections are examined for each sample. Static and structural parameters of bone formation and resorption are measured at a standardized site below the growth plate in the secondary spongiosa.

Urine is collected in metabolic cages. Urinary deoxypyridinoline is measured by ELISA and creatinine via a third party diagnostic laboratory. Other plasma markers are evaluated by ELISA included osteocalcin, bone sialoprotein, BMP (bone morphometric protein) and the catabolic marker carboxy-terminal-telopeptide.

The rats are sacrificed by exsanguination while under ether anesthesia. All animal data is obtained by blind measurements. Data are reported as mean±standard deviation (SD). The paired Student t-test is used to analyze values within the same group at baseline and after 6 weeks. ANOVA followed by the Newman-Keuls post-test is used to compare different groups. Linear regression between histomorphometric variables and non-invasive bone mass measurements is calculated and the Pearson test is applied. Statistical significance is set at P values lower than 0.05.

### REFERENCES

Cortez-Retamozo et al. (2004), Cancer Res 64: 2853-7.
Lipinsky, C A, et al. (2001), Adv Drug Deliv Rev 46: 3-26.
Nakashima, K. and de Crombrugghe, B., (2003), Trends Genet 19(8): 458-66

We claim:

1. A method for promoting osteogenesis in a population of vertebrate cells including osteoblast progenitor cells, comprising contacting osteoblast progenitor cells with an effective osteogenic stimulating amount of an LXR agonist.

2. A method for the treatment or prevention of an imbalance in bone homeostasis comprising administering an effective osteogenic stimulating amount of an LXR agonist to a subject suffering from or susceptible to said imbalance.

3. The method according to claim 2, wherein said imbalance in bone homeostasis is characterized by a reduction in the ratio of osteoblasts to osteoclasts in the bone tissue of said subject.

4. The method according to claim 3, wherein said LXR agonist promotes the differentiation of mesenchymal stem cells into osteoblasts in said subject's bone marrow thereby increasing the ratio of osteoblasts to osteoclasts.

5. A method according to claim 3, wherein said subject is susceptible to or suffering from hypocalcaemia (of malignancy), Paget's disease, rheumatoid arthritis, periodontal disease, focal osteogenesis occurring during skeletal metastases, Crouzon's syndrome, rickets, opsismodysplasia, pycnodysostosis/Toulouse-Lautrec disease, osteogenesis imperfecta or osteoprorosis.

6. The method of claim 5, wherein said treatment comprises administering to a subject suffering from osteoporosis.

7. The method according to claim 1, wherein said LXR agonist is a derivative of a diarylalkylaminoalkoxy2-phenyl acetic acid, a prodrug thereof, or a pharmaceutically acceptable salt, solvate or hydrate thereof.

8. The method according to claim 7, wherein said LXR agonist is 2-(3-(3-(N-(2-chloro-3-(trifluoromethyl)benzyl)-N-(2,2-diphenylethyl)amino)propoxy)phenyl)acetic acid, a prodrug thereof, or a pharmaceutically acceptable salt, solvate or hydrate thereof.

9. The method according to claim 1, wherein said LXR agonist is a N-(methyl)-N-[4-[2,2,2-trifluoro-1-hydroxy-1-(trifluoromethyl)ethyl]phenyl]-benzenesulfonamide, a prodrug thereof, or a pharmaceutically acceptable salt, solvate or hydrate thereof.

10. The method according to claim 1, wherein said LXR agonist is N-(2,2,2-trifluoroethyl)-N-[4-[2,2,2-trifluoro-1-hydroxy-1-(trifluoromethyl)ethyl]phenyl]-benzenesulfonamide, a prodrug thereof, or a pharmaceutically acceptable salt, solvate or hydrate thereof.

11. A method according to claim 4 wherein about 0.01 mg/kg to about 10 mg/kg of said LXR agonist administered from once to three times a day.

12. A method according to claim 11 wherein about 5 mg to about 1000 mg of said LXR agonist administered from once to three times a day.

13. A method according to claim 4 wherein said LXR agonist administered orally, transdermally, via inhalation, injection, nasally, rectally or via a sustained release formulation.

14. A method according to claim 13 wherein LXR agonist is administered to said patient for a period of time sufficient to reestablish normal bone homeostasis and thereafter to maintain such homeostasis.

15. A method of according to claim 13 wherein said LXR agonist is administered to a subject susceptible to the development of osteoporosis to prevent the onset of osteoporosis.

16. A bone homeostasis-promoting composition comprising an effective osteogenic stimulating amount of an LXR agonist in admixture with a pharmaceutically acceptable carrier.

17. The composition according to claim 16, wherein said LXR agonist is 2-(3-(3-(N-(2-chloro-3-(trifluoromethyl)benzyl)-N-(2,2-diphenylethyl)amino)propoxy)phenyl)acetic acid, a prodrug thereof, or a pharmaceutically acceptable salt, solvate or hydrate thereof.

18. The composition according to claim 16, wherein said LXR agonist is a N-(methyl)-N-[4-[2,2,2-trifluoro-1-hydroxy-1-(trifluoromethyl)ethyl]phenyl]-benzenesulfonamide, a prodrug thereof, or a pharmaceutically acceptable salt, solvate or hydrate thereof.

19. The method according to claim 16, wherein said LXR agonist is N-(2,2,2-trifluoroethyl)-N-[4-[2,2,2-trifluoro-1-hydroxy-1-(trifluoromethyl)ethyl]phenyl]-benzenesulfonamide, a prodrug thereof, or a pharmaceutically acceptable salt, solvate or hydrate thereof.

20. The method according to claim 16, wherein said LXR agonist is acetyl podocarpic dimer, a prodrug thereof, or a pharmaceutically acceptable salt, solvate or hydrate thereof.

21. A method according to claim 1, comprising the in vitro production of bone tissue, comprising applying osteoblast progenitor cells onto a substrate, contacting said cells with an effective osteogenic stimulating amount of an LXR agonist for a time sufficient to stimulate the generation of a matrix of bone tissue.
